SEQUENCE LISTING (123 sequences)

SEQ ID NO: 1 - DNA, 21 bp, Artificial Sequence
  Synthetic Oligonucleotide
tctggagcaggacagyttcga

SEQ ID NO: 2 - DNA, 21 bp, Artificial Sequence
  Synthetic Oligonucleotide
tgctgggcagcatgtaatcct

SEQ ID NO: 3 - DNA, 24 bp, Artificial Sequence
  Synthetic Oligonucleotide
attgaaacgaaataccctamyatg

SEQ ID NO: 4 - DNA, 19 bp, Artificial Sequence
  Synthetic Oligonucleotide
attccccagccagadcgtt

SEQ ID NO: 5 - DNA, 23 bp, Artificial Sequence
  Synthetic Oligonucleotide
ccgacgaccgaccccwayctgct

SEQ ID NO: 6 - DNA, 20 bp, Artificial Sequence
  Synthetic Oligonucleotide
gcgaagcgcgacanytccca

SEQ ID NO: 7 - DNA, 21 bp, Artificial Sequence
  Synthetic Oligonucleotide
gttagaaattttcgaygtcag

SEQ ID NO: 8 - DNA, 21 bp, Artificial Sequence
  Synthetic Oligonucleotide
atcatactcatcaaactccat

SEQ ID NO: 9 - DNA, 18 bp, Artificial Sequence
  Synthetic Oligonucleotide
cgggttgcgaaaacvatg

SEQ ID NO: 10 - DNA, 19 bp, Artificial Sequence
  Synthetic Oligonucleotide
acagatgctccarggtatg

SEQ ID NO: 11 - DNA, 623 bp, Aeromonas hydrophila
tgcttgttttcaaaggaaaattaaaagaacaccccagatgggaggtagaaaacgagctttatcgctttcgcaatcgcgtcttctccgatcgcctcggctgggatgtggaatcccaccgtggtctggagcaggacagtttcgatacccctgatacccactgggtgctgatcgaagacgaggaaggcctgtgcggctgcatccgtctgctcagctgtgccaaggattacatgctgcccagcatcttccccaccgccctcgccggtgaagccccgccgcgcagcaacgacgtgtgggagctgacccgcctcgccatcgatgccgaacgggctccccggctcggcaacggcatcagcgaactgacctgcatcatcttccgcgaggtctatgccttcgccaaggcgcaggggatccgagagctggttgccgtggtcagcctgccggtagagcggatcttccgccgcctcggtctgcccatcgaacggctcggtcaccgccaggcggtggatctgggcgccgtgcgcggggtggggatccgcttccatcttgatgagcggttcgcccgtgccgtcggccagcccctgcagggtgcctatgacgaggcgcgcgaactggtcacagaataa

SEQ ID NO: 12 - DNA, 614 bp, Aeromonas salmonicida
tcaaaggaaaattaaaagaacaccccagatgggaggtagaaaacgagctttatcgcttccgtaatcgcgtcttctccgatcgtctcggctgggatgtggagtctcaccgtggtctggagcaggacagcttcgacaccccggacacccattgggtgctgatcgaggacgaagaaggcctgtgtggctgcatccgtctgctcagttgcgcccaggattacatgctgcccagcatattccccaccgctctcgccggtgaggcgccgccacgcagcagcgatgtgtgggaactgactcgcctagccatcgacgccaaccgggcgccgcgcatgggcaacggggtgagcgagctgacctgcgtcatcttccgcgaggtttatgccttcgccagggcgaaggggatccgggaactggtcgccgtggtcagcctgccggtggaacgtatcttccgccgtctcggcctgcccatagagcgactcggtcaccgtcaagccgtggatctgggcgccgtgcgcggggtcggaatccgctttcatctggatgaacgtttcgcccgtgccgtcggccaccccatgcagggcgaatatgccgatgccagggaactggtcaccgagtaa

SEQ ID NO: 13 - DNA, 651 bp, Erwinia carotovora
atgttagagatatttgatgtaaatcacaccttgttgtcagaaacgaaatcaggagagctatttaccctcagaaaagagacgtttaaagatcgactgaattgggccgtgcaatgtactgatggaatggaatttgatcagtatgataataataacacgacttatctttttggcatcaaagataacaccgttatctgtagtttgagatttattgaaacaaaataccctaacatgattaccgggacattttttccttacttcaaggagatcaatatccctgaagggaattacctggagtctagtcggttttttgtcgataaatcgcgagcgaaagatattctcggcaatgaatacccaattagttcgatgttgtttctttcgatgattaattactcaaaggacaaaggctatgatggaatatatacgatagtgagtcaccctatgctgacgatattaaaacgatctggctggggaattcgcgtggtcgaacaaggtttgtcagaaaaggaagaaagagtctatttagtttttcttccggttgatgatgagaatcaggaagcgttggctcgtcgtattaaccgcagcgggacgtttatgagcaatgagttgaagcagtggcctctacgggttcctgctgctattgcacaggcttga

SEQ ID NO: 14 - DNA, 627 bp, Burkholderia cepacia
acagatccgaggacatccatgcagaccttcgttcacgaggaagggcggttgccacacgaactcgcggcggatctcgggcgctatcggcgccgcgtgttcgtcgagcagctcggttgggcgctcccgtcggcgaacgaaagtttcgagcgtgaccagttcgatcgcgacgataccgtctacgtgttcgcgcgaaacgccgacggcgacatgtgcggatgtgcgcgcctgctgccgacgacgcgcccgtatctgctgaagtcgctgttcgccgacctggtcgccgaagacatgccgctgccgcaatcggccgccgtctgggaattgtcgcgattcgcggcgaccgacgacgaaggcggtccgggcaacgccgaatgggccgtgcggccgatgctcgcggccgtcgtcgaatgcgcggcccagctcggggcgcggcagttgatcggcgtgacgttcgcgagcatggagcggctgttccgccggatcggcatacacgcgcaccgcgcaggcccgccgaagcaggtagatgggcgtctggtggtcgcgtgctggatcgacatcgatccgcaaacgtttgctgcgctaggaatcgagccggggcaagccgcccggcaagctatcgccgcctga

SEQ ID NO: 15 - DNA, 633 bp, Rhodobacter sphaeroides
atgatcttcattatcgactcgctcaacctccgcgagcacgcagacatcgtgaaggacatgttccggttgcgcaagcgcgtttttgcggaccgtctcggctgggacgttcagatttcgcagggcatggagcgcgaccgcttcgacgacctggatccggctcacgtcgtgagcgtggatgacgagggccgcgtggtcggctgcatgcgcctgcttcagacgacgggcccgcacatgctctccgacgtgttcagctcgatcctggacggagagcctccgctgcgcagcgcaactctctgggaagccacgcgcttctgtgtcgacaccgaccgcctggtttcgggccgcgcccgcaattcgatcgcctatgtcacgagcgaagtgatgatcggcgccttcgaattcgccatgtcggcgggtgtgaccgatgcggtcgccgtgatcgatccggtgatggaccgcgtgctgaagcggtcgggcaatgcgccgcagggctatgtcggcacgccgaagccgatgggcaaggtgacggctctggccgcgctgatggactgctcggaagagcgtgtgaagcgcatccgcgatttcgccggcatctaccacgacgtgacgcaaccgcagacggtcatcgcctga

SEQ ID NO: 16 - DNA, 651 bp, Enterobacter agglomerans
atgttagaaatttttgatgtcagttacaacgacctgacagaaagacgttcggaagacctttacaaattgaggaaaataacctttaaggatcgtctggattgggccgttaattgcagcaacgatatggagttcgacgagtttgataattcaggtacgcgctacatgctgggaatttatgataatcagctggtctgcagtgtgcgtttcattgatttgcgtttgccgaatatgattacgcatacgttccaacatttatttggtgacgtcaaattaccggagggtgattatatcgaatcaagccgtttttttgtcgataagaaccgagcgaaggcgttgctaggcagccggtacccgataagctatgttctctttttgtccatgattaattacgcgcgtcatcacggacacaccgggatttacaccattgtcagtcgcgccatgttaacgatcgctaaacgttcgggttgggagattgaggtgataaaagaagggtttgtcagtgaaaatgaacccatttatcttctgcgccttccgattgactgccataaccaacacctgctcgcaaaaaggatccgcgatcagtcagagtcaaatatcgcagcgctgtgccagtggcctatgtctttgacggtgacaccagaacaggtttaa

SEQ ID NO: 17 - DNA, 639 bp, Erwinia chrysanthemi
atgttagaaatatttgatgtgagttttagcttaatgtcaaataacaagctggatgaggtgtttacactacgcaaagatacatttaaggaccgtcttgactgggccgtcaactgtattaatgggatggagttcgacgagtatgataatgagcacaccacctaccttttaggcgtcaaagagggaaaggtgatttgcagcgtccggtttatagaaataaaataccccaatatgattaccggcacgttttactcttatttcgataacctcaagatccccgaagggaactatattgaatccagtcgtttttttgttgaccgtgatcgggtaagaaatttaattggcacccgcaatccggcatgtgtgacgttattcctcgcgatgattaattacgccagaaaataccattacgacggtatcctcacgattgtcagccacccgatgttgaccttgttgaaacgttcaggctggcgcatttccatcatccagcaagggctatcagaaaaacaggagcgcatttatttgctgcatctgcccacagatgacgacagccgacacgcgctgattgagcgcattactcaaatgacacaggccgagtccgagcagcttaagaccttgcctttactggttccactggcttga

SEQ ID NO: 18 - DNA, 633 bp, Erwinia stewartii
atgcttgaactgtttgacgtcagttacgaagaactgcaaaccacccgttcagaagaactttataaacttcgcaagaaaacatttagcgatcgtctgggatgggaagtcatttgcagtcagggaatggagtccgatgaatttgatgggcccggtacacgttatattctgggaatctgcgaaggacaattagtgtgcagcgtacgttttaccagcctcgatcgtcccaacatgatcacgcacacttttcagcactgcttcagtgatgtcaccctgcccgcctatggtaccgaatccagccgtttttttgtcgacaaagcccgcgcacgtgcgctgttaggtgagcactaccctatcagccaggtcctgtttttagcgatggtgaactgggcgcaaaataatgcctacggcaatatctatacgattgtcagccgcgcgatgttgaaaattctcactcgctctggctggcaaatcaaagtcattaaagaggctttcctgaccgaaaaggaacgtatctatttgctgacgctgccagcaggtcaggatgacaagcagcaactcggtggtgatgtggtgtcacgtacgggctgtccgcccgtcgcagtcactacctggccgctgacgctgccggtctga

SEQ ID NO: 19 - DNA, 654 bp, Erwinia carotovora
atgttagaaatattcgatgtaagctacacactactgtcggaaaaaaaatcggaagaattgtttacgcttaggaaagaaacgttcaaggataggctgaattgggcggtaaaatgtattaacgggatggaattcgatcagtatgatgatgataatgcgacttatcttttcggtgtagagggtgatcaggttatttgcagttctcggctaattgaaacgaaatatcctaatatgattactggaacgtttttcccttattttgaaaaaatagatattccggaagggaagtatatcgagtcgagccggttttttgtagataaagcgcggtcaaaaactattctaggaaattcttatcccgttagtacgatgttcttcttggcaacggtgaattactcaaagagtaaaggatatgatggtgtttatacgattgtcagtcatcctatgctcacaatactaaaacgttctggttggaaaatttcgattgttgaacagggtatgtcagaaaaacacgaaagggtttatctactttttttacctgtcgataatgaaagccaggatgtgctagttcgtcgtataaatcacaatcaggaatttgttgaaagtaagttgcgagagtggccactgtctttcgagcctatgactgaaccggtcggataa

SEQ ID NO: 20 - DNA, 639 bp, Erwinia chrysanthemi
atgttagaaatatttgatgtgagttttagcttaatgtcaaataacaagctggatgaggtgtttgcactacgcaaagggacatttaaggaccgtctggactggaccgtcaactgtattaacgggatggaatttgacgagtacgataatgaacacaccacctacttgttaggtgtcaaggaaggaaaaatcatttgtagtgtcagatttatagaaatgaaatatcccaacatgattactggcacatttttctcttatttcgacggcctcaacattcccgaagggaattatattgaatccagccgatttttcgtcgaccgcgatcgggtgagaaatttgattggcacccgtaatccagcttgcctgacattatttcttgccatgatcaattacgccagaaaatatcattatgatgggatactcacgattgtcagccacccgatgctgaccttgttaaaacgttcaggctggcgcatttccatcattcagcaggggttatcagaaaaacaggagaaaatttatctgctgcatctgcccacagacgatgaaagccgatacgcgctgatcgagcgcattacacggataaccaacgccgaatccgagcagcttacgaccttgcctttgctggtcccactggcttga

SEQ ID NO: 21 - DNA, 582 bp, Vibrio fischeri
atggctgtaatgataaaaaaatcggactttttgggcattccatcagaggagtatagaggtattcttagtcttcgttatcaggtatttaaacgaagactggagtgggacttggtaagtgaggataatcttgaatcagatgaatatgataactcaaatgcagaatatatttatgcttgtgatgatgcggaagaggtaaatggctgttggcgtttgttacctacaacgggtgattacatgttaaaaactgtttttcctgaattgctcggagatcaagtagccccaagagatccaaatatagtcgaattaagccgttttgctgtgggaaaaaatagctcaaaaataaataactctgctagtgaaataacaatgaaattgtttcaagctatatataaacacgcagttagtcaaggtattacagaatatgtaacagtaacatcaatagcaatagagcgatttctgaaacgtattaaagttccttgtcatcgcattggtgataaggagattcatttattaggtaatactagatctgttgtattgtctatgcctattaatgatcagtttagaaaagctgtatcaaattaa

SEQ ID NO: 22 - DNA, 664 bp, Pseudomonas aureofaciens
tcttgcaggtgccaagccggtacaagtcctctataaaactgcactccctttatccctaaggaaaacttcagtaatgcacatggaagagcacacactgaaccaaatgagcgacgagctgaaactcatgctcggccgctttcggcacgaacagttcgtcgagaaactcggatggcggctgccggcccatccgagccaggcaggttgtgaatgggatcaatacgacaccgagcacgcccgctacctcctggcgttcaatgaagaccgcgccatcgttggctgcgcccgcctgattcccaccacgttccccaacctgcttgaaggagtgttcggccatacctgcgccggggccccgcccaagcatccagccatctgggaaatgactcgcttcaccacccgcgaaccgcaactggcgatgccgttgttctggagaagcctcaagacagccagcctggcgggcgcagatgccatcgtcgggatcgtcaacagcaccatggagcgctattacaaaatcaacggcgtccactacgaacgactgggcccggtcacggtgcaccagaatgaaaaaatcctcgccatcaaactctcggcccaccgcgagcatcaccgcagcgcagtcgcaccgtcagccttcatgtccgacacattattgagagagaccgcttga

SEQ ID NO: 23 - DNA, 606 bp, Pseudomonas fluorescens
aaggaatcccctgccatgcacatggaagagcacgcgctgagcgcaatggacgacgagcttaaactcatgctgggtcgctttcggcatgaacaattcgtcgaaaaactagggtggcgattacctattccaccgcaccaggccggttatgaatgggatcaatacgacaccgagcacgcccgctacctcctggcgttcaacgagcatcgttcaatcgtcggctgcgcccggctgatacctaccacattccctaatctgctggagggtgtattcagccatgcctgcgcaggcgcaccacccaggcatcccgccatttgggaaatgacacgcttcaccacccgcgaaccgcaactggccatgcctttgttctggaaaaccctcaagaccgcgagcctggcgggtgccgatgccatcgtcggaatcgtcaacagcacgatggaacgctattacaagatcaacggggtcaagtacgaacgcctggggtcggttatcgaccatcagaacgaaaaaatcctcgccatcaaactctcggctcaccgcgaacaccaccgtggcgcccgcttaccttcaggcttcacgtctgaggcgttattggaagagactgcttga

SEQ ID NO: 24 - DNA, 681 bp, Pseudomonas syringae
atgtcgagcgggtttgagtttcagttagccagttacacgacgatgcccgttacgttgctggaaacgctatattccatgcgcaagaagattttttcagatcggcttgagtggaaagttcgtgtgagtcatgcatttgaatttgatgaatacgacaatgccgctaccacttaccttgtcggaagctggaacggggttccgctggccggactaaggctgatcaacacctgtgatccctacatgctggaaggcccattccgcagtttttttgactgccctgcgcccaaaaatgctgccatggcggagtcgagccggttctttgtcgacacggctcgggcacgctcgttgggtattctgcacgcaccactgaccgaaatgctgctgttctccatgcacaaccatgctgcgttgtccggtttgcaatcgatcatcacggtggtcagcaaggcaatggcgcggatcgtgcgcaagtccggatgggagcaccatgtgctgtcgaccggtgaagcatcgccaggcgagacagtgctgctgctggagatgccggtgacggccgacaaccatcagcggctgctcgggaatattgccctcagacagcctgtcacggatgacctgctgcgttggccgatagcgcttggcgtatcaggctccgcaccgcaagcctgcatgcacagcgctgcctga

SEQ ID NO: 25 - DNA, 606 bp, Pseudomonas aeruginosa
atgatcgaattgctctctgaatcgctggaagggctttccgccgccatgatcgccgagctgggacgctaccggcatcaggtcttcatcgagaagctgggctgggacgtggtctccacctccagggtccgcgaccaggagttcgaccagttcgaccatccgcaaacccgctacatcgtcgccatgggccgccagggtatctgcggttgtgcccgcctgttgccgacgaccgacgcctacctgctcaaggaagtcttcgcctacctgtgcagcgaaaccccgcccagcgatccgtcggtatgggagctttcgcgctacgccgccagcgcggcggacgatccgcaactggcgatgaagatattctggtccagcctgcaatgcgcctggtacctgggcgccagttcggtggtggcggtgaccaccacggccatggagcgctatttcgttcgcaacggcgtgatcctccagcgcctcggcccgccgcagaaggtcaagggcgagacgctggtcgcgatcagcttcccggcctaccaggagcgcggcctggagatgctgctgcgctaccacccggaatggctgcagggcgtaccgctgtcgatggcggtgtga

SEQ ID NO: 26 - DNA, 615 bp, Ralstonia solanacearum
atgcgtacattcgttcatggcggtggacggctgcccgaaggcatcgatgcggcgctggcgcactatcggcaccaggtctttgtggggcggctcggctggcaactgcccatggctgacggcaccttcgagcgcgatcagtacgatcgtgacgacacggtctacgtggtcgcccgggatgagggcgggactatctgcggttgcgcgcgcctgctgccgaccacgcggccctatctgctgaaggacgtctttgcgtctttgctcatgcacggcatgccgcctcccgaatcgccggaggtgtgggagctgtcccgcttcgcggcgcggtccggcgcgccttgcccgcggtccgggcgcgcggactgggccgtgcgcccgatgctggcctccgtcgtgcaatgcgcggcgcaacgcggagcgcggcggctgatcggcgcgaccttcgtcagcatggtccggctgttccgccgcatcggtgtgcgcgcccatcgggccggtccggtgcgatgcatcggcggcaggccggtcgtggcgtgctggatcgatatcgatgcatcgacctgtgctgcgctgggcatcccgagcgcgtccgccgcgccgggacctgtgctgcagtag

SEQ ID NO: 27 - DNA, 603 bp, Serratia liquefaciens
atgatagagctctttgacgtagactataatttgctgccagataacagatcgaaagaactgttctctttaagaaaaaaaacgtttaaagaccgtttagactggctcgttaattgtgaaaataacatggagtttgacgaatacgataatcgtcacgccacctatattttcggcacctaccaaaatcacgtcatttgcagtttgcgatttattgaaacgaaatacccaaacatgatcagcgatggcgtgttcgacacttattttaacgatataaagctccccgatggaaattacgttgaagccagcagattatttatagataaggcgcgcattcaagcacttcaactccatcaagcgccgattagcgccatgctttttctgtcgatgataaattatgcaagaaactgcggctacgaaggcatttacgccattatcagtcacccgatgcgcatcattttccaacgttccggttggcatatctcggtggtaaaaaccggatgttccgaaaagaataaaaatatttacctgatctacatgccgatagacgatgccaaccgaaacagattgctagcacgcatcaatcaacacgccaccaaaatgggataa

SEQ ID NO: 28 - DNA, 801 bp, Agrobacterium tumefaciens
gtgcagatctgcacaaccttgtcggcgtctggtcggcctcacccgctcgcatttcgcgccttggaacaccgactcggtagaatcacctgaggtgcaaatctgcacgtaggcaaacgcaccgtgagatgatttctgttcgcatgacctgttcatgcggagattacgatgcggatactcacagtttcgccggaccaatacgaacggtatcgaagcttcctcaaacagatgcaccgccttcgcgcgacggtgttcggtggccgtctcgaatgggacgtctccatcatcgctggggaagagcgcgaccaatacgataatttcaagccgagctaccttctggcgattactgacagtgggcgggtcgccggatgcgtcagacttcttccggcttgcgggcctacgatgctggagcagactttttcgcaactcctcgaaatgggctcgctcgcagcgcattccggaatggtagagagctctcgcttctgcgtcgacacctccctcgtctcacggagagatgcgagccagctgcacctggcgactcttaccttattcgccggtatcattgaatggtcgatggccagcggctacacggagatcgtcacggcgaccgatcttcgtttcgagcgcatcctgaagcgtgctggatggccgatgcgccggcttggtgaacccaccgcgatcggcaacaccattgccattgccggaagactgccagccgatcgtgccagcttcgagcaggtttgccctccgggctactattctatcccccggatcgacgtggcagcgatcaggagtgccgcgtga

SEQ ID NO: 29 - DNA, 582 bp, Vibrio anguillarum
atgactatttcaatttattcacatacctttcaaagtgtccctcaagctgattatgtgtcattgctgaagttacgctataaagttttttcgcaacgcttgcagtgggagctaaaaacaaatcgaggaatggagactgatgagtacgacgttccagaagcacactatttgtatgccaaagaggaacagggtcatttagtggggtgttggcgaattttgccaacgacgtcgcgttatatgcttaaagatactttttcggaattactcggtgtgcagcaagcacccaaagcaaaggagatttatgagctgagtcgttttgcggtcgataaagatcattcggcgcaattgggcggcgtgagtaatgttacgctgcagatgtttcagtcgctgtatcatcacgcccaacaatatcacatcaatgcctatgtaacggtcacatcggccagtgtggaaaaattgattaagcggatggggatcccatgcgaaagactcggtgataaaaaagtgcatcttttgggaagtacacgttctgtcgctttgcatattccaatgaatgaagcatatcgtgcaagtgtcaatgcataa

SEQ ID NO: 30 - DNA, 573 bp, Pseudomonas aeruginosa
gggctttccgccgccatgatcgccgagctgggacgctaccggcatcaggtcttcatcgagaagctgggctgggacgtggtctccacctccagggtccgcgaccaggagttcgaccagttcgaccatccgcaaacccgctacatcgtcgccatgggccgccagggcatctgcggttgtgcccgcctgctgccgacgaccgacgcctacctgctcaaggaagtcttcgcctacctgtgcagcgaaaccccgccgagcgatccgtcggtctgggagctttcgcgctacgccgccagcgcggcggacgatccgcaactggcgatgaagatattctggtccagcctgcaatgcgcctggtacctgggcgccagttcggtggtggcggtgaccaccacggccatggagcgctatttcgttcgcaacggcgtgatcctccagcgcctcggcccgccgcagaaggtcaagggcgagacgctggtcgcgatcagcttcccggcctaccaggagcgcggcctggagatgctgctgcgctaccacccggaatggctgcagcgtacgctgtcgatggcggtgtga

SEQ ID NO: 31 - DNA, 645 bp, Yersinia enterocolitica
atgttaaaactctttaacgtaaattttaataatatgccggaaaggaagttagacgagattttctcacttagagaaataacatttaaagatcgactggactggaaagtaacctgtattgatggcaaagaaagcgaccaatatgatgatgagaacactaattatatattaggaacaatagacgacactattgtttgcagtgttcgttttattgatatgaaatatccaaccatgattacagggccatttgctccttatttttctgatgtgagcttacctattgatggttttattgaatccagccgattctttgttgaaaaagcattagcaagagatatggtgggcaataacagttcccttagtactatactgtttcttgcgatggtgaattatgccagggatcgtgggcataaagggatactcacggtagtcagccgaggaatgtttatactactgaaacgctcaggttggaatattacggtattgaaccaaggtgaatcagagaaaaatgaagttatttatcttttacacttgggtattgataacgatagccagcaacaactgataaataaaatactgagagtacatcaagttgaaccaaaaactctcgaaacctggccgataattgtacctggtattattaaatag

SEQ ID NO: 32 - DNA, 651 bp, Yersinia ruckeri
atgttagaaattttcgatgtcagttatgaagaattaatggatatgaggtctgatgatctttacaggttaagaaagaaaacgtttaaagatcggcttcaatgggcagtgaactgtagtaatgacatggagtttgatgagtacgataatcctaacacacgatacctactcggaatttatggaaatcagcttatttgcagtgttcgttttattgaacttcaccgacctaatatgatcactcatacctttaacgctcagttcgacgatataatattaccggaaggtaactatatcgaatcgagtcgtttctttgttgataaatctggggcaaaaacactgttaggtaaccgctatcccatcagttatgtattatttcttgcggtgatcaattatacccgacatcacaagcataccggcatttatactattgttagccgtgccatgctaacaatactaaagcgttccggttggcaatttgacgtcattaaagaagcttttgttagcgaaaaagaacgtatctacctgctccgtcttccggttgataaacacaaccaggctctgttggcatcgcaggttaatcaggttctacagggttctgattcagcattgctggcttggcccatttcgctgcccgttataccagaactggtttaa

SEQ ID NO: 33 - DNA, 651 bp, Yersinia pseudotuberculosis
atgttagaaattttcgatgtcagatacgacgagttgaccgatatacggtctgaggacctgtataaattaaggaaaaagacgtttaaggatcgtcttaactgggaggtgaattgcagcaacggcatggagtttgatgaatatgataattcggatacacgctatctccttggtatttaccaaggtcaactaatctgcagtgtgcgttttatagaacttcacctgcctaatatgatcactcataccttcaatgcactcttcgatgacgtggcattacctaagaggggctatatagaatcgagccgtttcttcgttgataaaacccgtgccaagttgctttttggtaaccattaccccataagctacttattctttttatcgattattaattactcccgacataatggctacacaggtatttatactatcgtcagccgagcaatgttgaccatccttaaacgatcaggctggcaggttgaggtgattaaagaagcacatattaccgaaaaagaaaggatatacttactgcatctcccgatagatcgggacaatcaagcacggctgcttttacaggttaatcagcgtttgcaggatccatgttcagtattaagtacatggcctatatcgttgcccgttatgccagaatcggcttaa

SEQ ID NO: 34 - DNA, 519 bp, Vibrio harveyi
atgcctttattagacagcttcaccgtagaccacacgcgtatgaatgcaccagcggttcgtgtggctaaaacgatgcaaactccaaaaggagacaccatcacggtattcgacctacgtttcactgctccaaacaaagacatcctttctgagaaaggaattcatacattagagcatttgtacgcaggctttatgcgtaatcacctaaatggtgatagcgttgagatcattgatatctcaccaatggggtgccgtactggtttctacatgagcttgattggtacgccttcagagcagcaagtggctgacgcttggattgccgcgatggaagacgtactaaaagtagaaaaccaaaacaagatccctgagttgaacgaataccaatgtggtacagcagcgatgcactctctggatgaagcgaagcaaatcgcgaagaacattctagaagtgggtgtggcggtgaataagaatgatgaattggcactgccagagtcaatgctgagagagctacgcatcgactaa

SEQ ID NO: 35 - DNA, 492 bp, Salmonella typhi
aattcggatcataccggatgcaagcgccggcggtccgggttgcaaaaacgatgaacaccccgcatggcgacgcaatcacgtgtttgatctgcgtttttgcattccgaacaaagaagtgatgccggaaaaagggattcatacgcttgagcatctgtttgctggctttatgcgcgaccacctcaacggtaacggcgttgagattatcgatatctcgccgatgggctgccgcaccggcttttacatgagcctgattggcacgccggacgagcagcgtgttgccgacgcctggaaagcggcgatggcggatgtgctgaaagtgcaggatcaaaaccagatcccggagctgaacgtttaccagtgcggtacgtatcagatgcactcgctcagtgaagcgcaggacattgcccgtcatattctggagcgtgatgtgcgcgtgaacagcaataaagagctggcgctgccgaaagaaaaactgcaggaactgatatttag

SEQ ID NO: 36 - DNA, 519 bp, Vibrio cholerae
atgccattattagacagttttaccgtcgatcatactcgtatgaatgcaccggcggtgcgtgttgccaaaaccatgcaaaccccaaaaggggatacgattaccgtatttgatttgcgttttactatgccaaacaaagatatcttgtctgagcgcggtatccatactctagagcatctctacgcgggctttatgcgcaatcaccttaacggcagccaagtggagatcatcgatatttcaccaatgggttgccgtacaggtttctacatgagcttgattggtgcgccgacagaacagcaagtggcacaagcatggctagccgcaatgcaagatgtgttgaaagttgaaagccaagagcaaattcctgagctgaatgagtaccagtgcggcactgcggcgatgcactcgctcgaagaagccaaagcgattgcgaaaaacgtgattgcggcaggcatctcggttaaccgtaacgatgagttggcgctgcccgaatctatgctcaatgagctgaaggttcactaa

SEQ ID NO: 37 - DNA, 516 bp, Escherichia coli
atgccgttgttagatagcttcacagtcgatcatacccggatggaagcgcctgcagttcgggtggcgaaaacaatgaacaccccgcatggcgacgcaatcaccgtgttcgatctgcgcttctgcgtgccgaacaaagaagtgatgccagaaagagggatccataccctggagcacctgtttgctggttttatgcgtaaccatcttaacggtaatggtgtagagattatcgatatctcgccaatgggctgccgcaccggtttttatatgagtctgattggtacgccagatgagcagcgtgttgctgatgcctggaaagcggcaatggaagacgtgctgaaagtgcaggatcagaatcagatcccggaactgaacgtctaccagtgtggcacttaccagatgcactcgttgcaggaagcgcaggatattgcgcgtagcattctggaacgtgacgtacgcatcaacagcaacgaagaactggcactgccgaaagagaagttgcaggaactgcacatctag

SEQ ID NO: 38 - DNA, 504 bp, Haemophilus influenzae
atgccattacttgatagttttaaagtggatcacacaaaaatgaacgcacctgcagtacgcattgcaaaaacgatgctcacgccaaaaggcgataatattactgtttttgatttacgtttttgtattccaaacaaagaaattctttccccaaaaggcattcatacacttgaacatttatttgctggatttatgcgcgatcatttaaatggcgatagcatagaaattattgatatttctccgatgggatgtcgcacgggattttatatgtctttgattggcacaccaaatgaacagaaagtgtctgaggcttggttagcttcaatgcaagatgttttaggtgtacaagatcaagcttctattcctgaattaaatatctatcaatgcggaagctatacggaacattccttagaagatgcacacgaaattgccaaaaatgttatcgcacgcggtataggtgtaaataaaaatgaagatttgtcactcgataattccttattaaaatag

SEQ ID NO: 39 - DNA, 20 bp, Artificial Sequence
  Synthetic Oligonucleotide
catctgtttgctggctttat

SEQ ID NO: 40 - DNA, 18 bp, Artificial Sequence
  Synthetic Oligonucleotide
catctggcgyrccaatca

SEQ ID NO: 41 - DNA, 21 bp, Artificial Sequence
  Consensus Sequence
agacctcgtcctgtcaaagct

SEQ ID NO: 42 - DNA, 21 bp, Artificial Sequence
  Consensus Sequence
agacctcgtcctgtcraagct

SEQ ID NO: 43 - DNA, 21 bp, Aeromonas hydrophila
tctggagcaggacagtttcga

SEQ ID NO: 44 - DNA, 21 bp, Aeromonas salmonicida
tctggagcaggacagcttcga

SEQ ID NO: 45 - DNA, 23 bp, Artificial Sequence
  Synthetic Oligonucleotide
ggctgctggctggggatggacga

SEQ ID NO: 46 - DNA, 23 bp, Artificial Sequence
  Consensus Sequence
ggvtgstgbvhssgvwtrgacga

SEQ ID NO: 47 - DNA, 23 bp, Pseudomonas aeruginosa
ccgacgaccgacgcctacctgct

SEQ ID NO: 48 - DNA, 23 bp, Pseudomonas aeruginosa
ccgacgaccgacgcctacctgct

SEQ ID NO: 49 - DNA, 23 bp, Burkholderia cepacia
ccgacgacgcgcccgtatctgct

SEQ ID NO: 50 - DNA, 23 bp, Ralstonia solanacearum
ccgaccacgcggccctatctgct

SEQ ID NO: 51 - DNA, 23 bp, Pseudomonas aureofaciens
cccaccacgttccccaacctgct

SEQ ID NO: 52 - DNA, 23 bp, Pseudomonas fluorescens
cctaccacattccctaatctgct

SEQ ID NO: 53 - DNA, 20 bp, Artificial Sequence
  Consensus Sequence
cgcttcgcgctgtcgagggt

SEQ ID NO: 54 - DNA, 20 bp, Artificial Sequence
  Consensus Sequence
crcwtmgcncwkthragggt

SEQ ID NO: 55 - DNA, 20 bp, Pseudomonas aeruginosa
cgcatcgcgctttcgagggt

SEQ ID NO: 56 - DNA, 20 bp, Burkholderia cepacia
cgcttagcgctgttaagggt

SEQ ID NO: 57 - DNA, 20 bp, Ralstonia solanacearum
cgcttcgccctgtcgagggt

SEQ ID NO: 58 - DNA, 20 bp, Pseudomonas aureofaciens
cacttcgctcagtaaagggt

SEQ ID NO: 59 - DNA, 20 bp, Pseudomonas fluorescens
cacttcgcacagtaaagggt

SEQ ID NO: 60 - DNA, 21 bp, Artificial Sequence
  Consensus Sequence
caatctttaaaagctacagtc

SEQ ID NO: 61 - DNA, 21 bp, Artificial Sequence
  Consensus Sequence
crawcttkamaarctrcagtc

SEQ ID NO: 62 - DNA, 21 bp, Yersinia ruckeri
gttagaaattttcgatgtcag

SEQ ID NO: 63 - DNA, 21 bp, Enterobacter agglomerans
gttagaaatttttgatgtcag

SEQ ID NO: 64 - DNA, 21 bp, Erwinia stewartii
gcttgaactgtttgacgtcag

SEQ ID NO: 65 - DNA, 21 bp, Artificial Sequence
  Synthetic Oligonucleotide
tagtatgagtagtttgaggta

SEQ ID NO: 66 - DNA, 21 bp, Yersinia ruckeri
tagcatgagtagtttgaggta

SEQ ID NO: 67 - DNA, 21 bp, Yersinia pseudotuberculosis
tagtataagtagtttgaggta

SEQ ID NO: 68 - DNA, 21 bp, Enterobacter agglomerans
tagtttgagcagcttgaggta

SEQ ID NO: 69 - DNA, 21 bp, Erwinia stewartii
tagtttaagtagcctgaggta

SEQ ID NO: 70 - DNA, 24 bp, Artificial Sequence
  Synthetic Oligonucleotide
taactttgctttatgggattgtac

SEQ ID NO: 71 - DNA, 24 bp, Artificial Sequence
  Consensus Sequence
tawctwtrytttatgggattgtac

SEQ ID NO: 72 - DNA, 24 bp, Erwinia carotovora
attgaaacaaaataccctaacatg

SEQ ID NO: 73 - DNA, 24 bp, Erwinia carotovora
attgaaacgaaatatcctaatatg

SEQ ID NO: 74 - DNA, 24 bp, Erwinia chrysanthemi
atagaaatgaaatatcccaacatg

SEQ ID NO: 75 - DNA, 24 bp, Erwinia chrysanthemi
atagaaataaaataccccaatatg

SEQ ID NO: 76 - DNA, 24 bp, Serratia liquefaciens
attgaaacgaaatacccaaacatg

SEQ ID NO: 77 - DNA, 24 bp, Yersinia enterocolitica
attgatatgaaatatccaaccatg

SEQ ID NO: 78 - DNA, 19 bp, Artificial Sequence
  Consensus Sequence
taaggggtcggtcttgcaa

SEQ ID NO: 79 - DNA, 19 bp, Artificial Sequence
  Consensus Sequence
tahrvggtygghcthgcaa

SEQ ID NO: 80 - DNA, 19 bp, Erwinia carotovora
taaggggtcggtctagcaa

SEQ ID NO: 81 - DNA, 19 bp, Erwinia carotovora
taaaaggttggtcttgcaa

SEQ ID NO: 82 - DNA, 19 bp, Erwinia chrysanthemi
tacgcggtcggacttgcaa

SEQ ID NO: 83 - DNA, 19 bp, Serratia liquefaciens
tatacggttggccttgcaa

SEQ ID NO: 84 - DNA, 19 bp, Yersinia enterocolitica
tataaggttggactcgcaa

SEQ ID NO: 85 - DNA, 18 bp, Artificial Sequence
  Consensus Sequence
gcccaacgcttttgctac

SEQ ID NO: 86 - DNA, 18 bp, Artificial Sequence
  Consensus Sequence
gcvyamcgnttttgbtac

SEQ ID NO: 87 - DNA, 18 bp, Escherichia coli
cgggtggcgaaaacaatg

SEQ ID NO: 88 - DNA, 18 bp, Salmonella typhi
cgggttgcaaaaacgatg

SEQ ID NO: 89 - DNA, 18 bp, Vibrio cholerae
cgtgttgccaaaaccatg

SEQ ID NO: 90 - DNA, 18 bp, Vibrio harveyi
cgtgtggctaaaacgatg

SEQ ID NO: 91 - DNA, 18 bp, Haemophilus influenzae
cgcattgcaaaaacgatg

SEQ ID NO: 92 - DNA, 19 bp, Artificial Sequence
  Consensus Sequence
tgtctacgaggtcccatac

SEQ ID NO: 93 - DNA, 19 bp, Artificial Sequence
  Consensus Sequence
tvtyyacragdtyncatac

SEQ ID NO: 94 - DNA, 19 bp, Artificial Sequence
  Consensus Sequence
tgtccacgaggtcccatac

SEQ ID NO: 95 - DNA, 19 bp, Salmonella typhi
tgtctacgagttcgcatac

SEQ ID NO: 96 - DNA, 19 bp, Vibrio cholerae
tctctacgagatctcatac

SEQ ID NO: 97 - DNA, 19 bp, Vibrio harveyi
tgtttacgagattacatac

SEQ ID NO: 98 - DNA, 19 bp, Haemophilus influenzae
tatttacaagttcacatac

SEQ ID NO: 99 - DNA, 20 bp, Artificial Sequence
  Consensus Sequence
gtrrabawrcghccdaaata

SEQ ID NO: 100 - DNA, 20 bp, Escherichia coli
cacctgtttgctggttttat

SEQ ID NO: 101 - DNA, 20 bp, Salmonella typhi
catctgtttgctggctttat

SEQ ID NO: 102 - DNA, 20 bp, Vibrio cholerae
catctctacgcgggctttat

SEQ ID NO: 103 - DNA, 20 bp, Vibrio harveyi
catttgtacgcaggctttat

SEQ ID NO: 104 - DNA, 20 bp, Haemophilus influenzae
catttatttgctggatttat

SEQ ID NO: 105 - DNA, 18 bp, Artificial Sequence
  Consensus Sequence
gtagaccgcatggttagt

SEQ ID NO: 106 - DNA, 18 bp, Artificial Sequence
  Consensus Sequence
ghmddccrcryggttagt

SEQ ID NO: 107 - DNA, 18 bp, Escherichia coli
gtagaccgcatggttagt

SEQ ID NO: 108 - DNA, 18 bp, Salmonella typhi
gcaggccgcacggttagt

SEQ ID NO: 109 - DNA, 18 bp, Vibrio cholerae
gacagccgcgtggttagt

SEQ ID NO: 110 - DNA, 18 bp, Vibrio harveyi
gacttccgcatggttagt

SEQ ID NO: 111 - DNA, 18 bp, Haemophilus influenzae
gtaaaccacacggttagt

SEQ ID NO: 112 - DNA, 18 bp, Artificial Sequence
  false priming site in Vibrio group bacteria
gttttccgctgtgttagt

SEQ ID NO: 113 - DNA, 18 bp, Artificial Sequence
  false priming site in Vibrio group bacteria
accgaaatacgcgttagt

SEQ ID NO: 114 - DNA, 614 bp, Artificial Sequence
  Consensus Sequence
tcaaaggaaaattaaaagaacaccccagatgggaggtagaaaacgagctttatcgctttcgcaatcgcgtcttctccgatcgcctcggctgggatgtggaatcccaccgtggtctggagcaggacagtttcgatacccctgatacccactgggtgctgatcgaagacgaggaaggcctgtgcggctgcatccgtctgctcagctgtgccaaggattacatgctgcccagcatcttccccaccgccctcgccggtgaagccccgccgcgcagcaacgacgtgtgggagctgacccgcctcgccatcgatgccgaacgggctccccggctcggcaacggcatcagcgaactgacctgcatcatcttccgcgaggtctatgccttcgccaaggcgcaggggatccgagagctggttgccgtggtcagcctgccggtagagcggatcttccgccgcctcggtctgcccatcgaacggctcggtcaccgccaggcggtggatctgggcgccgtgcgcggggtggggatccgcttccatcttgatgagcggttcgcccgtgccgtcggccagcccctgcagggtgcctatgacgaggcgcgcgaactggtcacagaataa

SEQ ID NO: 115 - DNA, 614 bp, Artificial Sequence
  Consensus sequence
tcaaaggaaaattaaaagaacaccccagatgggaggtagaaaacgagctttatcgcttycgyaatcgcgtcttctccgatcgyctcggctgggatgtggartcycaccgtggtctggagcaggacagyttcgayacccckgayacccaytgggtgctgatcgargacgargaaggcctgtgyggctgcatccgtctgctcagytgygccmaggattacatgctgcccagcatmttccccaccgcyctcgccggtgargcsccgccrcgcagcarcgaygtgtgggarctgacycgcctmgccatcgaygccramcgggckccscgsmtsggcaacggsrtsagcgarctgacctgcrtcatcttccgcgaggtytatgccttcgccarggcgmaggggatccgrgarctggtygccgtggtcagcctgccggtrgarcgkatcttccgccgyctcggyctgcccatmgarcgrctcggtcaccgycargcsgtggatctgggcgccgtgcgcggggtsggratccgcttycatctkgatgarcgkttcgcccgtgccgtcggccascccmtgcagggygmmtatgmcgakgcsmgsgaactggtcacmgartaa

SEQ ID NO: 116 - DNA, 600 bp, Artificial Sequence
  Consensus sequence
atgttagaaatatttgatgtaaattataccttgttgtcagaaaagaaatcggaagagctgtttaccctcagaaaagagacgtttaaagatcgactggactgggccgtgaaatgtattaatggaatggaatttgaccagtatgataatgataacacgacttatctttttggcatcaaagatgacaacgttatttgcagtttgcgatttattgaaacgaaataccctaacatgattaccgggacatttttcccttatttcaaggagatcaatatccctgaagggaattatattgaatccagccggttttttgtcgataaagcgcgagcgaaagatattctcggcaatgaatatccaattagttcgatgttgtttcttgcgatgattaattacgcaaggaacaaaggctatgatggaatatacacgattgtcagtcaccctatgctgacgatattaaaacgttctggctggggaatttccgtggtcgaacaaggtttgtcagaaaaagaagaaagaatttatctagtttatctgccgattgatgatgagagccaggaagcgctggttcgtcgtattaaccgcagcgggacgttgatgagataa

SEQ ID NO: 117 - DNA, 599 bp, Artificial Sequence
  Consensus Sequence
atgwtararmtmttyraygtrrryywyavhhwdhtgycrrawavvarryyrrrhgarntdttydcnythmgvraarddacrttyaargaymgdytdraytggvhvgtnmmhtgtrhwrayrrvawrgarwkygaysartaygatratvrdmayrcbamytayhtdtthgghrymdwysrnrrhmmnrtbrtytgyagykybmgdytwatwgawayraaataycchamyatgatyashggvvyrttykhyhcttayttydmnrrnvtvrrbhthccbrwwgrdrrbtwyvtbgarkcbagymgdtthttyrthgahmrnkmdykvdydmrarmwhtdvwnskmmmynvhdvbycvvywwgynybayrytnttytbkcvaygrtnaattaykcmarrrrhhrhsrnyayrahggnrthywyrcsrtwrtsagycrhssdatgykbayvhtdytvmaacghtchggytggvrhatyhstnvavtnvavmmvggdnddtcmgaraarvanrarvdnrtytayytdntnywyhtdssbryhgayraygmnarycrrnahvnrytrvyhvvdmrhathmhnmrvvdmvnvavbhhdwdbvvrhvd

SEQ ID NO: 118 - DNA, 534 bp, Artificial Sequence
  Consensus Sequence
gggctttccgccgccatgatcgccgagctgggacgctaccggcatcaggtcttcgtcgagaagctgggctgggagctctccaccgccagggtccgcttcgagtgcgaccagttcgaccatgagcaaacccgctacatcgtcgccatgggccgccagggtatctgcggttgtgcccgcctgttgccgacgaccgacccctacctgctcaaggaagtcttcgcctacctgtgcagcgaagccccgcccagcgatccggcggtatgggagctgtcgcgcttcgccgcacgcgtccgcaactggcgatgcagatgttctggtccagcctgcaatgcgcctggcacctgggcgccagttcggtggtcggggtgaccaccacgaccatggagcgctatttcgttcgcaacggcgtgatcctccagcgcctcggcccgccgaagaatcaagggcgagacgctggtcgcgtcccggaccgggagcgcggcctggagatgtgctgcgctagcactcgggatggctgcagcgtacgctatatgc

SEQ ID NO: 119 - DNA, 536 bp, Artificial Sequence
  Consensus Sequence
svrhtknvmsmmgvvmtbrhnsysrhkctsgsncrctwycggcrysrvswvttyrtsgrrmrrctvggntggsvrsybycvwyskmnvvvgdmmsbkwvgarykbgaycartwcgaymvysmssahrcsskctacvtsbtsgcshkvrgmvsvyvgbdhatskkygghtgygcscgsctghtdccbacsacvbdsscbwayctgctbrarkvnstvttyrssbmynysbkcrbvsrvrvmmyrccsmvsvatccvkmsrtntgggarhtkwcncgmtwcrcsrcmcrcrhscssamykggcsrtgmvkwrtytskssdvmrbcstsvarwsmgcsdssywvsbsggngcvvrkbvsntsrtsgsvrysryswhcrbsrssatggwvcgsywktwcvdnmksawcggbrtvmdsbhssancgvsyvggbycgsybmhsvwwhvrbrrbvrdmhrvtsstsgcstsyyggaycrvbadcrmdscvyvrmsvhbykywsckyyrssmhycvnrhbbgvbnmvkykbvvgvdrbykvtgm

SEQ ID NO: 120 - DNA, 633 bp, Artificial Sequence
  Consensus Sequence
atgttagaaattttcgatgtcagttacgaagaattgatggatatgcggtctgatgatctttacaaattaagaaagaaaacgtttaaagatcgtcttcaatgggcagtgaattgcagtaatgacatggagtttgatgagtatgataatcctgatacacgatacctactcggaatttatgaaaatcagcttatttgcagtgttcgttttattgaacttcaccgacctaatatgatcactcatacctttaacgctcagttcgatgatgtaatattaccggaaggtgatatcgaatcgagccgtttctttgttgataaatcccgggcaaaaacgctgttaggtaaccgctaccccatcagctatgtattattttttgcgatgatcaattatacccgacatcacgagcacaccggcatttatactattgtcagccgtgccatgttaacaatactaaagcgttccggttggcaatttgacgtcattaaagaagcttttgttagcgaaaaagaacgtatctacctgctccgtcttccggttgataaacacaaccaggctctgctggcatcgcaggttaatcagcttctacagggttctgattcagcattgctggcttggcccatttcgctgcccgttata

SEQ ID NO: 121 - DNA, 633 bp, Artificial Sequence
  Consensus Sequence
atgytwgaamtkttygaygtcagwtayramgavytrmhvrmhabvmgktcdgadgahctktayarrytdmgvaarawracvtttarvgatcgkctkvrhtgggmvgtbawytgyagymabgrhatggagtyygaygartwygatrrkycnrryacrcghtaymtnctbggwatytrysrwrrwcarytdrtbtgcagygtdcgtttyayhrrhytbsryykdccbaayatgatyackcayacbttymansmhydvttyrrygayrtvrhmytrccbrmvdrkrrtaymgaatcvagycgtttyttygtygayaardmysgngcvmrddyrytkytwggyrrscrbtayccbatmagyyabkthytvtttytdkcsrtkrtbaaytrbdcscrwmatmayrvvyayrsmrrbatytayacbatygtyagycghgcvatgytramvathsyhamdcghtcnggytggsardtyravgtsatwaaagargsdywyvtbasygaaaadgaamsbathtayytdstsmbbctbccrrywgryhrvsayraymarsmncdrytssbwdndvrkrtbvdbbmdsdkhyrsrskvwhmdnnykymgyrbtvhbbvmntggccbmtdwckytgmcsgtbwbr

SEQ ID NO: 122 - DNA, 480 bp, Artificial Sequence
  Consensus Sequence
cagtcgatcataccggatgaaagcacctgcggttcgggtggcgaaaacgatgcacaccccaaaaggcgacacaatcacgtgtttgatctgcgttttgtattccaaacaaagaaagtgatgtcagaaaaagggattcataccctggagcatctgtttgctggctttatgcgcaatcaccttaacggtaatggcgtagagattatcgatatctcgccaatgggctgccgcaccggtttttacatgagcttgattggtacgccagatgagcagcaagttgctgatgcctggatagcggcaatggaagatgtgctgaaagtgcaagatcaaaatcagatccctgagctgaacgtctaccagtgcggcacttacccgatgcactcgctggaggaagcgcaggatattgcgaataacattctggaacgtggtgtacgcgtcaacagcaatgaagaattggcactgccgaaatcgatgctgaaggag

SEQ ID NO: 123 - DNA, 478 bp, Artificial Sequence
  Consensus Sequence
mmktvgaycayacnrdatgvahgcrccdgcrgtncgbrtkgcnaaaacvatgmwmacbccrmawggvgayrmnatyacgtdttygayytrcgyttysyrykccraacaaagahrtbhtkycnsmrmrmggnatycatacnytdgarcayytvtwygcdgghtttatgcgyraycayythaayggyrryvrhrtdgaratyatygatatytcdccratgggntgycgyacngghttytayatgwsyytgattggyrcrccddmhgarcagmrwgtkchsangchtgghwwgcbkcratgsmrgaygtdytrrrwgtdsarrrycarrmbhmdatycckgarytraayrwntaycartgygghasnkmnvmgrwrcaytcbytvvrdgawgcvmavsmnatygcsmrdmryrtkmtngmrsbngryrtvbsbrtnaaymrnaayrawgadytgkcrctssmnradkmnwwrytvmrdkar

(19) United States
(12) Patent Application Publication
(10) Pub. No.: US 2004/0023254 A1
(43) Pub. Date: Feb. 5, 2004

(54) Method to assess quorum sensing potential of microbial communities

(76) Inventors: Jeffry J. Fuhrmann, Lincoln University, PA (US); James A. Romesser, Kennett Square, PA (US)

Correspondence Address: William J. McNichol, Jr., Esq., Reed Smith LLP, 2500 One Liberty Place, 1650 Market Street, Philadelphia, PA, 19103, US

(21) Appl. No.: 10/338,110

(22) Filed: Jan. 7, 2003

Related U.S. Application Data
(60) Provisional application No. 60/346,531, filed on Jan. 8, 2002.

(51) Int. Cl. C12Q 1/68; C12P 19/34

(52) U.S. Cl. 435/6; 435/91.2

(57) ABSTRACT

A method for detecting quorum sensing potential in a sample is provided comprising extracting nucleic acid from a sample comprising at least one type of microorganism; performing a polymerase chain reaction on the nucleic acid using oligonucleotide primers, wherein each of the primers comprises at least 15 nucleobases and anneal to a consensus sequence of a lux gene and homologs thereof, wherein the first eight nucleobases at the 3′ end of the primers comprise a match to said consensus sequence with optional degeneracy at positions 4-8 from the 3′ end of the primers; separating the amplified DNA fragments; and determining whether any amplified DNA fragment corresponds to the size of a predicted product related to quorum sensing potential. Methods of amplifying fragments of lux genes and homologs thereof are also provided as well as isolated nucleic acid fragments of lux and kits for performing PCR reactions.

### CROSS-REFERENCE TO RELATED APPLICATIONS

The benefit of U.S. Provisional Application Serial No. 60/346,531, filed Jan. 8, 2002, is hereby claimed. That application is incorporated herein by reference in its entirety.

### BACKGROUND OF THE RELATED ART

1. Field of the Invention

The invention pertains to a method of detecting quorum sensing potential in microorganisms. Specifically, the invention pertains to the use of the polymerase chain reaction to detect quorum sensing potential in bacteria. Bacterial taxa present in a community may be manipulated based on information obtained from the detection method.

2. Background of the Related Art

Several genera of bacteria have been shown to “communicate” with one another via quorum sensing. Quorum sensing is a means by which bacteria communicate their presence to surrounding bacteria so as to regulate cell density and specific population-dependent events. Quorum sensing systems rely on the constitutive expression, at low levels, of an autoinducer molecule that triggers the expression of a particular set of genes when its concentration in solution reaches some threshold level. This threshold can only be attained when sufficient numbers (a “quorum”) of the bacteria are present in a localized area such that the combined rates of degradation and diffusional dilution of the autoinducer are less than its rate of production. In this way, expenditure of energy for the expression of the genes involved occurs only under conditions in which their production has a reasonable likelihood of conferring a significant ecological advantage to the bacteria producing the autoinducer.

A number of microbial processes are known to rely on quorum sensing as a means of controlling gene expression. These include bioluminescence by marine bacteria inhabiting light organs of deep sea-dwelling organisms such as squid, pathogenesis by plant and animal (including human) disease-causing bacteria, and biofilm formation by biofouling bacteria.

Bacteria differ in the type of autoinducer produced. At a broad scale, Gram-negative and Gram-positive bacteria typically produce acyl homoserine lactones (AHSLs) and peptides, respectively. Differences also occur within these groupings. For example, a given Gram-negative bacterium may produce one or multiple acyl lactones (ACLs) including N-(3-oxohexanoyl)-L-homoserine lactone (OHHL), N-(3-oxododecanoyl)-L-homoserine lactone (OdDHL), N-butanoyl-L-homoserine lactone (BHL), and N-hexanoyl-L-homoserine lactone (HHL). These differences in the acyl chain affect the biological properties of the autoinducers, and allow for specificity to a particular bacterial genotype or group, genetic control based on interacting AHSLs, and autoinducer crosstalk and interferences among bacterial genotypes (Swift et al, 1996).

The best-characterized AHSL-based quorum sensing system is that described originally for bioluminescence in Vibrio fischeri (Kaplan and Greenberg, 1985). It consists of two genes, luxI and luxR, whose expression is responsible for the production and detection of autoinducer, respectively. Subsequent research revealed the presence of genes homologous to luxI and luxR in many other bacteria which regulate genes involved in numerous other microbial processes, as alluded to above.

Bacteria are a diverse group of organisms and the ability to detect quorum sensing potential among such a diverse group of organisms in a rapid and efficient manner is not known. Thus, there is a need in the art for a rapid, reliable method of detecting quorum sensing potential in bacteria.

### SUMMARY OF THE INVENTION

The invention provides methods of detecting quorum sensing potential in microorganisms comprising (a) extracting nucleic acid from a microorganism; (b) performing a polymerase chain reaction using the nucieic acid wherein the polymerase chain reaction comprises a first oligonucleotide primer and a second oligonucleotide primer, wherein each primer is comprised of at least 15 nucleobases; wherein the primers anneal to a consensus sequence of a lux gene and homologs thereof; wherein the first eight nucleobases at the 3′ end of the primers comprise a match to the consensus sequence with optional degeneracy at positions 4-8 from the 3′ end of the primers; wherein the polymerase chain reaction results in amplified DNA fragments; (c) separating the amplified DNA fragments; and (d) determining whether any amplified DNA fragment corresponds to the size of a predicted product related to quorum sensing potential, thereby detecting quorum sensing potential of the microorganism.

The invention also provides methods of detecting quorum sensing potential in microorganisms from an environmental sample comprising (a) extracting nucleic acid from the environmental sample; (b) performing a polymerase chain reaction using the nucleic acid wherein the polymerase chain reaction comprises a first oligonucleotide primer and a second oligonucleotide primer, wherein each primer is comprised of at least 15 nucleobases; wherein the primers anneal to a consensus sequence of a lux gene and homologs thereof; wherein the first eight nucleobases at the 3′ end of the primers comprise a match to the consensus sequence with optional degeneracy at positions 4-8 from the 3′ end of the primers; wherein the polymerase chain reaction results in amplified DNA fragments; (c) separating the amplified DNA fragments; and (d) determining whether any amplified DNA fragment corresponds to the size of a predicted product related to quorum sensing potential, thereby detecting quorum sensing potential of microorganisms in an environmental sample.

In the embodiments of method of the invention the lux gene may be luxI luxR, luxS gene or homologs thereof.

The microorganisms that may be tested using the methods of the invention may be bacteria, preferably Gram-negative bacteria. However, the methods of the invention may be applied to other microorganisms (including Gram-positive bacteria) if the microorganisms have homologous genes to lux.

The amplified fragments formed in the PCR reactions may be separated by any means known in the art, such as on gels (e.g., agarose gels and polyacrylamide gels) or by liquid chromatography.

In some embodiments of the methods of the invention, the primers used comprise the sequences of SEQ ID NO: 1 and SEQ ID NO: 2. In other embodiments of the methods of the invention, the primers used comprise the sequences of SEQ ID NO: 3 and SEQ ID NO: 4. In other embodiments of the methods of the invention, the primers used comprise the sequences of SEQ ID NO: 5 and SEQ ID NO: 6. In other embodiments of the methods of the invention, the primers used comprise the sequences of SEQ ID NO: 7 and SEQ ID NO: 8. In other embodiments of the methods of the invention, the primers used comprise the sequences of SEQ ID NO: 9 and SEQ ID NO: 10.

The invention also provides compositions comprising (a) a nucleic acid sequence comprising a lux gene or homolog thereof; (b) at least two oligonucleotide primers, wherein each of said primers comprises at least 15 nucleobases, wherein said primers anneal to a consensus sequence of a lux gene and homologs thereof; wherein the first eight nucleobases at the 3′ end of said primers comprise a match to said consensus sequence with optional degeneracy at positions 4-8 from the 3′ end of said primers; and (c) at least one enzyme having DNA polymerase activity.

The compositions may further comprise a mixture of deoxynucleotide triphosphates.

The compositions may further comprise pairs of primers having sequences selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; and SEQ ID NO: 9 and SEQ ID NO: 10. Alternatively, the compositions may comprise at least one pair of primers selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; and SEQ ID NO: 9 and SEQ ID NO: 10.

The invention also provides a method of amplifying a fragment of a lux gene or homolog thereof comprising (a) extracting nucleic acid from a sample comprising at least one type of microorganism, said nucleic acid comprising a lux gene or homolog thereof; and (b) performing a polymerase chain reaction using the nucleic acid wherein the polymerase chain reaction comprises a first oligonucleotide primer and a second oligonucleotide primer, wherein each of the oligonucleotide primers comprises at least 15 nucleobases, wherein the oligonucleotide primers anneal to a consensus sequence of a lux gene and homologs thereof; wherein the first eight nucleobases at the 3′ end of the oligonucleotide primers comprise a match to the consensus sequence with optional degeneracy at positions 4-8 from the 3′ end of said primers; wherein the polymerase chain reaction results in amplified DNA fragments.

In some embodiments of the method of the invention, the sample is a culture of microorganisms comprising at least one type of microorganism. In other embodiments of the method of the invention, the sample is a sample taken from the environment.

The method of amplifying the fragment may further comprise isolating an amplified fragment of the lux gene or homolog thereof.

The method of amplifying the fragment may comprise using oligonucleotide primer pairs selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10

The invention also provides isolated DNA fragments made by the method of the invention.

The invention further provides kits for the amplification of a portion of a lux gene, or homolog thereof comprising, in separate containers, (a) a polymerase, (b) a plurality of deoxynucleotide triphosphates; (c) a first oligonucleotide primer; and (d) a second oligonucleotide primer; wherein each of said primers comprises at least 15 nucleobases, wherein said primers anneal to a consensus sequence of a lux gene and homologs thereof; and wherein the first eight nucleobases at the 3′ end of said primers comprise a match to said consensus sequence with optional degeneracy at positions 4-8 from the 3′ end of said primers.

The first oligonucleotide primer and second oligonucleotide primers may be SEQ ID NO: 1 and SEQ ID NO: 2, respectively; SEQ ID NO: 3 and SEQ ID NO: 4, respectively; SEQ ID NO: 5 and SEQ ID NO: 6, respectively; SEQ ID NO: 7 and SEQ ID NO: 8, respectively; or SEQ ID NO: 9 and SEQ ID NO: 10, respectively.

### BRIEF DESCRIPTION OF THE FIGURES

FIG. 1 shows a cluster dendrogram of the DNA sequence similarities among luxI homologs submitted to Genbank. Grouping codes: A=Aeromonas-like, E=Erwinia-like, P=Pseudomonas-like, and Y =Yersinia-like.

FIG. 2 shows an electrophoretic separation on an agarose gel of amplified fragments obtained from PCR reactions using degenerate primers derived from an alignment of luxI homologs of bacteria in the Pseudomonas group.

FIG. 3 shows an electrophoretic separation on an agarose gel of amplified fragments obtained from PCR reactions using degenerate primers derived from an alignment of luxI homologs of bacteria in the Aeromonas group (F122D2 (SEQ ID NO: 1) and R240ND (SEQ ID NO: 2)) and DNA extracted from selected bacterial cultures. White arrow indicates products of expected size (˜119 bp) from homologous DNAs. Annealing temperature used was 47° C., but this was changed in subsequent experiments to 60° C. to eliminate weak bands in lanes 1, 4, and 6.

FIG. 4 shows an electrophoretic separation on an agarose gel of amplified fragments obtained from PCR reactions using degenerate primers derived from an alignment of luxI homologs of bacteria in the Pseudomonas group (F295D4 (SEQ ID NO: 5) and R398D6 (SEQ ID NO: 6)) and DNA extracted from selected bacterial cultures. White arrow indicates products of expected size (101±˜3 bp) from homologous DNAs. Annealing temperature used was 60° C.

FIG. 5A-C shows results of partial sequencing of PCR products presumed to be specific luxI homologs. A=P. aeruginosa PG 201, B=B. cepacia K56-2, and C=A. salmonicida 1102. Within each subfigure, A-C, the first sequence is that obtained from Genbank (expected sequence), the second is that obtained for the PCR product, and the third is the minority consensus for the first two sequences. Bullets indicate mismatches or gaps; boldface letters were edited manually using the original sequencing chromatograms.

FIG. 6 shows electrophoretic separation on an agarose gel of PCR products obtained using mixtures of DNA extracted from selected bacterial cultures and two sets of primers (Aeromonas and Pseudomonas) derived from an alignment of associated luxI homologs. Expected product sizes are ˜119 and ˜101 bp for the Aeromonas and Pseudomonas primers, respectively.

FIG. 7 shows electrophoretic separation on an agarose gel of PCR products obtained using Aeromonas (A) and Pseudomonas (B) primers to amplify DNA extracted from samples taken from various equipment and locations within a paper mill. Expected product sizes are ˜119 and ˜101 bp for the Aeromonas and Pseudomonas primers, respectively.

FIG. 8 shows an alignment of luxI homologs from Aeromonas hydrophilia (ahyl) (SEQ ID NO: 11), and A. salmonicida (asaI) (SEQ ID NO: 12) with the minority consensus sequence (SEQ ID NO: 110) and majority consensus sequence (SEQ ID NO: 109).

FIG. 9A-B shows an alignment of luxI homologs from Erwinia carotovara (carI) (SEQ ID NO: 13), E. carotovara (expI), E. chrysanthemi (echI) (SEQ ID NO: 17), Serratia liquifaciens (swrI) (SEQ ID NO: 27), and Yersinia eneterocolitica (yenI) (SEQ ID NO: 31) with the minority consensus sequence (SEQ ID NO: 1 12) and majority consensus sequence (SEQ ID NO: 111).

FIG. 10A-C shows an alignment of luxI homologs from Pseudomonas aeruginosa (rhlI) (SEQ ID NO: 25), P. aeruginosa (vsmI) (SEQ ID NO: 30), Burkholderia cepacia (cepI) (SEQ ID NO: 14), Ralstonia solanacearum (solI) (SEQ ID NO: 26), P. aureofaciens (phzI) (SEQ ID NO: 22), and P. fluorescens (phzI) (SEQ ID NO: 23) with the minority consensus sequence (SEQ ID NO: 114) and majority consensus sequence (SEQ ID NO: 113).

FIG. 11A-B shows an alignment of luxI homologs from Yersinia ruckeri (yukI) (SEQ ID NO: 32), Y. pseudotuberculosis (ybtI) (SEQ ID NO: 33), Enterobacter agglomerans (eagI) (SEQ ID NO: 16), and Erwinia stewartii (esaI) (SEQ ID NO: 18) with the minority consensus sequence (SEQ ID NO: 116) and majority consensus sequence (SEQ ID NO: 115).

FIG. 12A-B shows an alignment of luxS homologs from Escherichia coli (SEQ ID NO: 37), Salmonella typhi (SEQ ID NO: 35), Vibrio cholerae (SEQ ID NO: 36), V. harveyi (SEQ ID NO: 34), and Haemophilus influenza (SEQ ID NO: 38) with the minority consensus sequence (SEQ ID NO: 117) and majority consensus sequence (SEQ ID NO: 116).

The words used in this specification have the meaning that would be attributed to those words by one skilled in the art, unless specifically defined herein. In the event that a conflict between a definition specifically defined herein and that understood by persons of ordinary skill in the art, the definition of the word or phrase as specifically taught herein shall control. Headings used herein are merely for convenience, and are not to be construed as limiting in any way.

### DETAILED DESCRIPTION OF THE INVENTION

Standard reference works setting forth the general principles of recombinant DNA technology known to those of skill in the art include Ausubel et al., CURRENT PROTOCOLS IN MOLECULAR BIOLOGY, John Wiley & Sons, New York, 1998 Molecular Cloning: A Laboratory Manual (3rd ed.) Sambrook, J. & D. Russell, Eds. Cold Spring Harbor Laboratory Press, Cold Spring Harbor, N.Y. (2001); Kaufinan et al., Eds., HANDBOOK OF MOLECULAR AND CELLULAR METHODS IN BIOLOGY AND MEDICINE, CRC Press, Boca Raton, 1995; McPherson, Ed., DIRECTED MUTAGENESIS: A PRACTICAL APPROACH, IRL Press, Oxford, 1991; and THE POLYMERASE CHAIN REACTION, Mullis, K. B., F. Ferre, and R. A. Gibbs, Eds., Birkhauser, Boston, 1994.

As used herein, “DNA” refers to deoxyribonucleic acid in its various forms as understood in the art, such as genomic DNA, cDNA, isolated nucleic acid molecules, vector DNA, chromosomal DNA. “Nucleic acid” refers to DNA or RNA in any form. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA molecules. “Isolated” nucleic acid removed from its native environment, such that it is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, is generally substantially free of other cellular material or culture medium when produced by recombinant techniques, or of chemical precursors or other chemicals when chemically synthesized.

As used herein the term “nucleobase” refers to a nucleotide that may be incorporated into a polymer of nucleotides. Nucleobases may be, for example, adenosine (A), thymidine (T), cytidine (C), guanosine (G), and inosine (I), and modified forms thereof. When an oligonucleotide primer is degenerate at a particular position, the degeneracy is designated as follows: Y=C+T; D=A+T+G; M=A+C; W=A+T, R=A+G; S=C+G; K=G+T; H=A+C+T; V=A+G+C; B=T+G+C; and N=A+G+C+T.

As used herein “nucleobase sequence” refers to a sequence of consecutive nucleobases.

As used herein, “anneal” refers to specific interaction between strands of nucleotides wherein the strands bind to one another substantially based on complementarity between the strands as determined by Watson-Crick base pairing. It is not necessary that complementarity be 100% for annealing to occur.

As used herein, “amplifying” refers to enzymatically increasing the amount of a specific nucleotide sequence in a polymerase chain reaction.

As used herein “denaturation” refers to the separation of nucleotide strands from an annealed state.

As used herein, “microorganism” is meant to include any of the phylogenetic domains of bacteria having lux homologs. The present invention is particularly effective to detect quorum sensing potential of Gram-negative cocci, and Gram negative straight, curved and helical/vibroid and branched rods.

The Gram-negative cocci include, but are not limited to, Halococcus, Mobiluncus, Moraxella species (including M catarrhalis), Neisseria species (including N. gonorrheae and N. meningitidis), and Veillonella.

The Gram-negative straight, curved, helical/vibrioid and branched rods include, but are not limited to, Acetobacter, Acinetobacter, Aeromonas, Agrobacterium, Alcaligenes, Aquaspirillum, Azotobacter, Bacteroides species (including B. fragilis), Bartonella, Bordetella species (including B. pertussis), Brucella, Burkholderia species (including B. cepacia), Calymmatobacterium granulomatis, Campylobacter species (including C. jejuni), Capnocytophaga, Caulobacter, Chromobacterium violaceum, Citrobacter, Comamonas, Edwardsiella, Eikenella, Enterobacter, Erwinia, Escherichia species (including E. coli), Flavobacterium species (including F. meninosepticum), Francisella species (including F. tularensis), Fusobacterium species (including F. nucleatum), Gardnerella species (including G. vaginalis), Gluconobacter, Haemophilus species (including H. influenzae and H. ducreyi), Hafnia, Helicobacter species (including H. pylori), Herpetosiphon, Klebsiella species (including K. pneumoniae), Kluyvera, Legionella species (including L. pneumophila), Leptotrichia, Morganella, Nitrobacter, Nitrosomonas, Pasteurella species (including P. multocida), Pectinatus, Photobacterium, Porphyromonas species (including P. gingivalis), Proteus species (including P. mirabilis), Providencia, Pseudomonas species (including P. aeruginosa, P. mallei, P. pseudomallei and P. solanacearum), Rahnella, Rhizobium, Salmonella, Serratia, Shigella, Spirillum, Streptobacillus species (including S. moniliformis), Vibrio species (including V. cholerae and V. vulnificus), Wolinella, Xanthobacter, Xenorhabdus, Yersinia species (including Y. pestis and Y. enterocolitica), Zanthomonas and Zymomonas.

Other Gram-negative bacteria include sulfur-oxidizing bacteria including, but not limited to, Thiobacillus species (including T ferroxidans); sulfur or sulfate-reducing bacteria including, but not limited to, Desulfovibrio; spirochetes including, but not limited to, Treponema species (including T. pallidum, T pertenue, T. hyodysenteriae and T. denticola), and Borrelia species (including B. burgdorferi and B. recurrentis); and rickettsias including, but not limited to, Cowdria, Ehrlichia, Neorickettsia, and Rickettsia.

Although the precise conditions of the Polymerase Chain Reaction (PCR) are not essential to the method of the invention and are well-known to those of ordinary skill in the art, a brief, general discussion is set forth herein. One of ordinary skill in the art, armed with the present disclosure and the knowledge of PCR in the art may make modifications of the basic PCR procedures using routine experimentation to optimize results for the particular use.

Those of ordinary skill in the art are well acquainted with PCR as a method of amplifying fragments of DNA based on repeated, cyclic phases of denaturation of DNA, annealing of oligonucleotide primers to complementary portions of the denatured strands of DNA and extension of the primers in the 5′ to 3′ direction through DNA polymerase driven polymerization of deoxynucleotide triphosphates (dNTPs) to form the complement of the denatured strand of DNA.

The denaturation step is generally accomplished through heating the PCR reaction to a temperature at which the strands of target DNA separate, or denature. Generally, the reactions are heated to at least about 94° C.

Often, the first round of denaturation is conducted for a longer period of time than subsequent denaturation phases. For example, the first denaturation may be conducted for up to 5 minutes or more. Typically, the first denaturation is conducted for less than 5 minutes. Subsequent denaturation phases are typically conducted for less than 2 minutes. In some embodiments, the denaturation phases are conducted for less than 1 minute. In other embodiments, the denaturation phases are conducted for 30 seconds or less. In other embodiments, the denaturation is conducted for as short a duration as 1 second.

The annealing phase of PCR is permitted to occur at a lower temperature than the denaturation phase. Typically, a temperature is selected that is lower than the calculated melting temperature of the oligonucleotide primers selected for the PCR reaction. Preferably, a temperature of about 5° C. below the calculated melting temperature is used. The melting temperature (Tₘ) for a given oligonucleotide primer may be calculated by any method known in the art, for example by using the formula: Tm=(number of A and T in oligonucleotide sequence x 2° C.)+(number of G and C in oligonucleotide sequence x 2° C.). In some embodiments, the temperature of the annealing phase is from about 37° C. to about 65° C. In other embodiments, the annealing phase is conducted at a temperature of about 50° C. to 60° C.

The duration of the annealing phase is not particularly limited. The duration may be as short as one second and may be as long as several minutes. In some embodiments, annealing is conducted for 30 seconds to about 2 minutes. In other embodiments, annealing is conducted for about 30 seconds to about 1 minute.

The extension phase of PCR is permitted to occur at any temperature that supports polymerization below a temperature that would cause the strands of DNA to denature. Typically, a temperature is chosen near to the temperature at which the polymerase has optimum processivity (where the enzyme incorporates dNTPs at the highest rate). For example, many polymerases currently used for PCR reactions have an optimum processivity rate at about 72° C. The extension phase temperature may be, for example, from about 25° C. to about 80° C. Preferably, the extension phase is conducted from about 37° C. to about 75° C. More preferably, the extension phase is conducted at a temperature of about 60° C. to about 72° C.

The duration of the extension phase is sufficient to allow the enzyme to incorporate dNTPs to form the complement of the target fragment. The duration depends on the size of the fragment to be amplified, the processivity rate of the enzyme, the buffer conditions of the enzyme and the temperature at which the extension phase is conducted. One of ordinary skill in the art will be able to adjust the duration of the extension phase in accordance with these parameters to suit the needs of the particular PCR. In some embodiments, a final extension phase is conducted for a longer period of time. In some embodiments this may be as long as 4 minutes or more.

In standard PCRs, cycles of denaturation, annealing and extension are performed providing exponential amplification of target sequences. Typically at least 20 cycles of PCR are performed. In some embodiments, 20-40 cycles are performed. In other embodiments, 25-35 cycles are performed.

In each PCR to amplify a DNA fragment associated with quorum sensing potential, the reactions comprise buffered water, a DNA polymerase, any cofactors required for polymerase activity (such as MgCl₂), an oligonucleotide primer that anneals to the (−) strand of DNA comprising a consensus sequence of a lux gene, an oligonucleotide primer that anneals to the (+) strand of DNA comprising a consensus sequence of a lux gene, dNTPs, and a sample of the microorganism DNA to be tested.

To design oligonucleotide primers that are suitable for use in the method of the invention, a lux gene and homologs thereof (available in sequence databases such as GenBank) may be aligned and consensus sequences identified. The oligonucleotide primers that are useful in the method of the invention are generally at least about 15 nucleobases in length. In some embodiments, the oligonucleotide primers are 15-40 nucleobases in length. In other embodiments, the oligonucleotide primers are 20-35 nucleobases in length. In other embodiments, the oligonucleotide primers are 25-30 nucleobases in length. The oligonucleotide primers suitable for use in the method of the invention comprises a match of the consensus sequence at the first three bases at the 3′ end of the primer (the three 3′ most nucleobases). The oligonucleotide primers may optionally have degeneracies in their sequence as compared with the sequence of the consensus sequence, however, it is preferable that the degeneracy of the oligonucleotide primer sequence be low, especially towards the 3′ end of the primer. In some embodiments, the degeneracy of the oligonucleotide primer sequence is within the base positions 4-8 from the 3′ end, and the remainder of the primer sequence is non-degenerate and based on the majority consensus of the alignment. In other embodiments, the degeneracy is within positions 4-6 from the 3′ end.

In some embodiments, the oligonucleotide primers suitable for use in the method of the invention comprise the sequences of SEQ ID NO: 1 and SEQ ID NO: 2. In other embodiments, the oligonucleotide primers comprise the sequences of SEQ ID NO: 3 and SEQ ID NO: 4. In other embodiments, the oligonucleotide primers comprise the sequences of SEQ ID NO: 5 and SEQ ID NO: 6. In other embodiments, the oligonucleotide primers comprise the sequences of SEQ ID NO: 7 and SEQ ID NO: 8. In some embodiments more than one pair of the following primers are used in the same PCR reaction: SEQ ID NO: 1 and SEQ ID NO: 2 (Aeromonas group); SEQ ID NO: 3 and SEQ ID NO: 4 (Erwinia group); SEQ ID NO: 5 and SEQ ID NO: 6 (Pseudomonas group); SEQ ID NO: 7 and SEQ ID NO: 8 (Yersinia group); SEQ ID NO: 9 and SEQ ID NO: 10 (Vibrio group).

Compositions of the invention for amplification of bacterial DNA comprise buffered water to support activity of DNA polymerase, including any cofactors (such as magnesium chloride), a DNA polymerase, and one or more pairs of oligonucleotide primers to amplify fragments of the various bacterial groups: SEQ ID NO: 1 and SEQ ID NO: 2 (Aeromonas group); SEQ ID NO: 3 and SEQ ID NO: 4 (Erwinia group); SEQ ID NO: 5 and SEQ ID NO: 6 (Pseudomonas group); SEQ ID NO: 7 and SEQ ID NO: 8 (Yersinia group); SEQ ID NO: 9 and SEQ ID NO: 10 (Vibrio group). In further embodiments, the compositions additionally comprise dNTPs. Bacterial DNA may be added to the compositions of the invention and PCR reactions may be performed.

The invention also encompasses methods of amplifying portions of bacterial lux homologs. In one embodiment the method of amplifying a fragment of a lux gene or homolog thereof comprises (a) extracting nucleic acid from a microorganism or environmental sample comprising at least one type of microorganism, wherein the nucleic acid comprises a lux gene or homolog thereof; and (b) performing a polymerase chain reaction using the nucleic acid wherein the polymerase chain reaction comprises a first oligonucleotide primer and a second oligonucleotide primer, wherein each of the primers comprises at least 15 nucleobases, wherein the primers anneal to a consensus sequence of a lux gene and homologs thereof; wherein the first eight nucleobases at the 3′ end of the primers comprise a match to the consensus sequence with optional degeneracy at positions 4-8 from the 3′ end of the primers; wherein the polymerase chain reaction resultsin amplified DNA fragments. In certain embodiments, the method of the invention uses primers having the sequences of SEQ ID NO: 1 and SEQ ID NO: 2 to amplify a portion of the luxI gene, or homolog thereof in the bacterial nucleic acid. The expected product size is 119 nucleobases. Thus, the invention also comprises isolated nucleic acid fragments comprising a portion of an Aeromonas group luxI gene produced by the method of the invention using primers having sequences of SEQ ID NO: 1 and SEQ ID NO: 2. The isolated nucleic acid fragments are homologous to luxI and comprise the sequences of SEQ ID NO: 1 and SEQ ID NO: 2 as their 5′ and 3′ ends, respectively. The isolated nucleotide fragments also hybridize to luxI under stringent hybridization conditions. As used herein “stringent hybridization conditions” refers to conditions under which a primer or oligonucleotide (such as an amplified fragment specifically hybridizes to its target sequence). Stringent conditions, as used herein, refers to hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C.

In other embodiments, the method of the invention uses primers having the sequences of SEQ ID NO: 3 and SEQ ID NO: 4 to amplify a portion of the luxI gene, or homolog thereof in the bacterial nucleic acid. The expected product size is 266-269 nucleobases. Thus, the invention also comprises isolated nucleic acid fragments comprising a portion of an Erwinia group luxI gene produced by the method of the invention using primers having sequences of SEQ ID NO: 3 and SEQ ID NO: 4. The isolated nucleic acid fragments are homologous to luxI and comprise the sequences of SEQ ID NO: 3 and SEQ ID NO: 4 as their 5′ and 3′ ends, respectively. The isolated nucleotide fragments also hybridize to luxI under stringent hybridization conditions.

In other embodiments, the method of the invention uses primers having the sequences of SEQ ID NO: 5 and SEQ ID NO: 6 to amplify a portion of the luxI gene, or homolog thereof in the bacterial nucleic acid. The expected product size is 98-104 nucleobases. Thus, the invention also comprises isolated nucleic acid fragments comprising a portion of an Pseudomonas group luxI gene produced by the method of the invention using primers having sequences of SEQ ID NO: 5 and SEQ ID NO: 6. The isolated nucleic acid fragments are homologous to luxI and comprise the sequences of SEQ ID NO: 5 and SEQ ID NO: 6 as their 5′ and 3′ ends, respectively. The isolated nucleotide fragments also hybridize to luxI under stringent hybridization conditions.

In other embodiments, the method of the invention uses primers having the sequences of SEQ ID NO: 7 and SEQ ID NO: 8 to amplify a portion of the luxI gene, or homolog thereof in the bacterial nucleic acid. The expected product size is 142 nucleobases. Thus, the invention also comprises isolated nucleic acid fragments comprising a portion of an Yersinia group luxI gene produced by the method of the invention using primers having sequences of SEQ ID NO: 7 and SEQ ID NO: 8. The isolated nucleic acid fragments are homologous to luxI and comprise the sequences of SEQ ID NO: 7 and SEQ ID NO: 8 as their 5′ and 3′ ends, respectively. The isolated nucleotide fragments also hybridize to luxI under stringent hybridization conditions.

In other embodiments, the method of the invention uses primers having the sequences of SEQ ID NO: 9 and SEQ ID NO: 10 to amplify a portion of the luxS gene, or homolog thereof in the bacterial nucleic acid. The expected product size is 266 nucleobases. Thus, the invention also comprises isolated nucleic acid fragments comprising a portion of an Vibrio group luxS gene produced by the method of the invention using primers having sequences of SEQ ID NO: 9 and SEQ ID NO: 10. The isolated nucleic acid fragments are homologous to luxS and comprise the sequences of SEQ ID NO: 9 and SEQ ID NO: 10 as their 5′ and 3′ ends, respectively. The isolated nucleotide fragments also hybridize to luxS under stringent hybridization conditions.

The following examples are merely illustrative of the invention, and are not intended to limit the scope of the disclosure or any claim.

### EXAMPLES

### Materials and Methods

Cultures, Sequences, and Environmental Samples

All cultures were obtained from the American Type Culture Collection (Bethesda, Md, www.atcc.org/) unless otherwise noted (Table 1). Working and archival cultures were stored at 4 and −80 C., respectively. Gene sequences were obtained from GenBank (www.ncbi.nlm.nih.gov/entrez/query.fcgi?db=Nucleotide); corresponding accession numbers appear in Table 1. Environmental samples of biofilms taken from various paper mills were obtained and stored at −20° C.

TABLE 1
Identifications and corresponding luxI homologs and Genbank accession
codes for bacterial cultures used to develop primers for assessment of
quorum sensing potentials.

| | luxI | GenBank | Culture and |
|---|---|---|---|
| Species | homolog | Accession No. | sources |
| Aeromonas hydro- | ahyI | X89469 | A1, |
| phila | | | S. Swift (U. |
| | | | Nottingham) |
| Aeromonas | asaI | U65741 | NCTC 1102, |
| salmonicida | | | S. Swift (U. |
| | | | Nottingham) |
| Agrobacterium | traI | L22207 | none |
| tumefaciens | | | |
| Bacillus cereus | na^{a} | na | Hercules isolate |
| | | | (M. Brancieri) |
| Bacillus subtilis | na | na | ATCC 23059 |
| Burkholderia | cepI | AF019654 | ATCC 25416^{b} |
| cepacia | | | |
| Burkholderia | cepI | AF019654 | K56-2, |
| cepacia | | | P. Sokol (U. |
| | | | Calgary) |
| Enterobacter | eagI | X74300 | ATCC 12985^{b} |
| agglomerans | | | |
| Erwinia caro- | carI | X74299 | GS101, |
| tovora | | | S. Swift (U. |
| | | | Nottingham) |
| Erwinia chrys- | expI | X96440 | none |
| anthemi | | | |
| Erwinia stew- | esaI | L32183 | none |
| artii | | | |
| Pseudomonas aeru- | rhlI (vmsI) | M59425 | ATCC 10145^{b} |
| ginosa | | (U15644) | |
| Psueodmonas aeru- | rhlI (vmsI) | M59425 | DSM2659 |
| ginosa | | (U15644) | (PG201)^{c}, P. |
| | | | Sokol (U. Cal- |
| | | | gary) |
| Pseudomonas | phzI | L33724 | ATCC 13985^{b} |
| chloroaphis | | | |
| (aureofaciens) | | | |
| Pseudomonas | phzI | L48616 | ATCC 13525^{b} |
| florescens | | | |
| Pseudomonas | psyI | AF110468 | none |
| syringae pv. | | | |
| tabaci | | | |
| Ralstonia sol- | solI | AF021840 | unknown^{d}, |
| anacearum | | | M. Schell (U. |
| | | | Georgia) |
| Serratia | swrI | U2283 | MG1, |
| liquefaciens | | | S. Molin (U. |
| | | | Denamrk) |
| Streptococcus | na | na | ATCC 25175 |
| mutans | | | |
| Vibrio anguill- | vanI | U69677 | none |
| arum | | | |
| Vibrio fischeri | luxI | M96844 | ATCC 7744^{b} |
| Yersinia entero- | yenI | X76082 | ATCC 49397^{b} |
| colitica | | | |
| Yersinia ruck- | yukI | AF079135 | ATCC 29473^{b} |
| eri | | | |

^{a}na, not applicable
^{b}Culture is not the source of GenBank sequence but is of the same species.
^{c}Original culture for vmsI/U15644 was not available.
^{d}Strain identification was not supplied with culture.

Primer Development

Initial alignment and cluster analysis of the homolog sequences were performed using Clustal version 1.7 available online at the EBI website (circinus.ebi.ac.uk:6543/cgi-bin/clustalw.cgi). Further alignments and primer design were performed using Primer Premier version 5.00 software (Premiere Biosoft International, Palo Alto, Calif., www.premierbiosoft.com/). Primers were purchased from Life Technologies, Inc., (which has since merged with Invitrogen, Carlsbad, Calif., www.invitrogen.com/). The sense and antisense primers and alignments for various bacterial groups and the predicted properties of the primers for the Aeromonas group, the Pseudomonas group, the Yersinia group, the Erwinia group, and the Vibrio groups was as follows:

A. Aeromonas group

Sequence Primer Premier alignment
(Sense Primer)

| | | |
|---|---|---|
| | 5′ TCTGGAGCAGGACAGYTTCGA 3′ | (SEQ ID NO:1) |
| | \|\|\|\|\|\|\|\|\|\|\|\|\|\|\| \|\|\|\|\| | |
| Maj. Con. | 3′ (122) AGACCTCGTCCTGTCAAAGCT (142) 5′ | (SEQ ID NO:36) |
| Min. Con. | 3′ (122) AGACCTCGTCCTGTCRAAGCT (142) 5′ | (SEQ ID NO:37) |
| Aeromonas ahyI | 5′ (199) TCTGGAGCAGGACAGTTTCGA (142) 3′ | (SEQ ID NO:38) |
| Aeromonas asaI | 5′ (113) TCTGGAGCAGGACAGCTTCGA (133) 3′ | (SEQ ID NO:39) |

(Antisense Primer)

| | | |
|---|---|---|
| | 5′ TGCTGGGCAGCATGTAATCCT 3′ | (SEQ ID NO:2) |
| | \|\|\|\|\|\|\|\|\|\|\|\|\|\|\|\|\|\|\|\|\| | |
| Maj. Con. | 3′ (220) ACGACCCGTCGTACATTAGGA (240) 5′ | (SEQ ID NO:2) |
| Min. Con. | 3′ (220) ACGACCCGTCGTACATTAGGA (240) 5′ | (SEQ ID NO:2) |
| Aeromonas ahyI | 3′ (220) ACGACCCGTCGTACATTAGGA (240) 5′ | (SEQ ID NO:2) |
| Aeromonas asaI | 3′ (211) ACGACCCGTCGTACATTAGGA (231) 5′ | (SEQ ID NO:2) |

Properties

| | | | | | | | | |
|---|---|---|---|---|---|---|---|---|
| | | | | | | | | Ta |
| | | | Tm | | ΔG | Activity | | Opt |
| Rating | Seq. No. | Length | (° C.) | GC % | (kcal/mol) | (μg/OD) | Degeneracy | (° C.) |

| | | | | | | | | | |
|---|---|---|---|---|---|---|---|---|---|
| Sense | 87 | 122 | 21 | 0.0 | 54.8 | −39.6 | 31.6 | 2 | — |
| Antisense | 82 | 240 | 21 | 62.9 | 52.4 | −42.0 | 33.3 | 1 | — |
| Product 0 | — | 119 | 88.9 | 58.0 | — | — | — | 37.2 | |

Product Size

| | |
|---|---|
| Aeromonas (ahyI) | 119 bp |
| Aeromonas (asaI) | 119 bp |

Secondary Structure of Sense Primer

| | |
|---|---|
| 1. | Most stable dimer (ΔG = −7.8 kcal/mol (3′ dimer)) |

| | |
|---|---|
| 5′ TCTGGAGCAGCAGGACAGYTTCGA 3′ | (SEQ ID NO:1) |
| \|\|\|\| | |
| 3′ AGCTTYGACAGGACGAGGTCT 5′ | (SEQ ID NO:1) |
| No false priming sites found. | |

Secondary Structure of antisense primer

| | |
|---|---|
| 1. | Most stable hairpin (ΔG = −2.2 kcal/mol) |

| | | |
|---|---|---|
| | GGTCGT 5′ | |
| | \|\|\|\|\| | |
| | GCAGCATGTAATCCT 3′ | (SEQ ID ID:2) |
| 1. | Most stable dimer (ΔG = −8.6 kcal/mol) | |
| | 5′ TGCTGGGCAGCATGTAATCCT 3′ | (SEQ ID NO:2) |
| | \|\|\|\|\| \|\|\|\|\| | |
| | 3′ TCCTAATGTACGACGGGTCGT 5′ | (SEQ ID NO:2) |
| 2. | (ΔG = −5.4 kcal/mol) | |
| | 5′ TGCTGGGCAGCATGTAATCCT 3′ | (SEQ ID NO:2) |
| | \|\| \|\|\|\| \|\| | |
| | 3′ TCCTAATGTACGACGGGTCGT 5′ | (SEQ ID NO:2) |
| | No false Priming sites found. | |

Secondary Structure or primer pair

| | |
|---|---|
| 1. | Most stable cross dimer (ΔG = −7.2 kcal/mol) (3′ cross dimer) |

| | | |
|---|---|---|
| | 5′ TCTGGAGCAGGACAGYTTCGA 3′ | (SEQ ID NO:1) |
| | \|\|\|\| | |
| | 3′ TCCTAATGTACGACGGGTCGT 5′ | (SEQ ID NO:2) |
| 2. | (ΔG = −6.7 kcal/mol) | |
| | 5′ TCTGGAGCAGGACAGYTTCGA 3′ | (SEQ ID NO:1) |
| | \| \|\|\|\| | |
| | 3′ TCCTAATGTACGACGGGTCGT 5′ | (SEQ ID NO:2) |
| 3. | (ΔG = −5.6 kcal/mol) (3′ cross dimer) | |
| | 5′ TCTGGAGCAGGACAGYTTCGA 3′ | (SEQ ID NO:1) |
| | \|\|\| \| \| | |
| | 3′ TCCTAATGTACGACGGGTCGT 5′ | (SEQ ID NO:2) |

B. Pseudomonad group

Sequence Primer Premier alignment
(Sense Primer)

| | | |
|---|---|---|
| | 5′ CCGACGACCGACCCCWAYCTGCT 3′ | (SEQ ID NO:5) |
| | \|\|\|\|\|\|\|\|\|\|\|\|\|\|\| \| \|\|\|\|\| | |
| Maj. Con. | 3′ (295) GGCTGCTGGCTGGGGATGGACGA (317) 5′ | (SEQ ID NO:40) |
| Min. Con. | 3′ (295) GGVTGSTGBVHSSGVWTRGACGA (317) 5′ | (SEQ ID NO:41) |
| P. aeruginosa rhlI | 5′ (220) CCGACGACCGACGCCTACCTGCT (242) 3′ | (SEQ ID NO:42) |
| P. aeruginosa vsmI | 5′ (190) CCGACGACCGACGCCTACCTGCT (212) 3′ | (SEQ ID NO:43) |
| Burkholderia cepI | 5′ (232) CCGACGACGCGCCCGTATCTGCT (254) 3′ | (SEQ ID NO:44) |
| Ralstonia solI | 5′ (220) CCGACCACGCGGCCCTATCTGCT (236) 3′ | (SEQ ID NO:45) |
| P. aureofaciens phzI | 5′ (220) CCCACCACGTTCCCCAACCTGCT (315) 3′ | (SEQ ID NO:46) |
| P. fluorescens phzI | 5′ (220) CCTACCACATTCCCTAATCTGCT (257) 3′ | (SEQ ID NO:47) |

(Antisense Primer)

| | | |
|---|---|---|
| | 5′ GCGAAGCGCGACAIYTCCCA 3′ | (SEQ ID NO:6) |
| | \|\|\|\|\|\|\|\|\|\|\|\|\| \|\|\|\|\| | |
| Maj. Con. | 3′ (379) CGCTTCGCGCTGTCGAGGGT (398) 5′ | (SEQ ID NO:48) |
| Min. Con. | 3′ (379) CRCWTMGCNCWKTHRAGGGT (398) 5′ | (SEQ ID NO:49) |
| P. aeruginosa rhlI | 5′ (298) CGCATCGCGCTTTCGAGGGT (317) 3′ | (SEQ ID NO:50) |
| P. aeruginosa vsmI | 5′ (268) CGCATCGCGCTTTCGAGGGT (287) 3′ | (SEQ ID NO:50) |
| Burkholderia cepI | 5′ (316) CGCTTAGCGCTGTTAAGGGT (335) 3′ | (SEQ ID NO:51) |
| Ralstonia solI | 5′ (298) CGCTTCGCCCTGTCGAGGGT (317) 3′ | (SEQ ID NO:52) |
| P. aureofaciens phzI | 5′ (371) CACTTCGCTCAGTAAAGGGT (390) 3′ | (SEQ ID NO:53) |
| P. fluorescens phzI | 5′ (313) CACTTCGCACAGTAAAGGGT (332) 3′ | (SEQ ID NO:54) |

+TC,Properties

| | | | | | | | | |
|---|---|---|---|---|---|---|---|---|
| | | | | | | | | Ta |
| | | | Tm | | ΔG | Activity | | Opt |
| Rating | Seq. No. | Length | (° C.) | GC % | (kcal/mol) | (μg/OD) | Degeneracy | (° C.) |

| | | | | | | | | | |
|---|---|---|---|---|---|---|---|---|---|
| Sense | 100 | 295 | 23 | 0.0 | 67.4 | −48.8 | 34.2 | 4 | — |
| Antisense | 82 | 398 | 20 | 0.0 | 62.5 | −45.3 | 33.1 | 8 | — |
| Product | 84 | — | 104 | 91.1 | 63.5 | — | — | — | 38.8 |

Product Size

| | |
|---|---|
| P. aeruginosa (rhlI) | 98 |
| P. aeruginosa (vsmI) | 98 |
| Burkholderia (cepI) | 104 |
| Ralstonia (solI) | 104 |
| P. aureofaciens (phzI) | 98 |
| P. fluorescens (phzI) | 98 |

Secondary Structure of Sense Primer

No hairpins found.
No false priming sites found.

Secondary Structure of antisense primer

| | |
|---|---|
| | No hairpins found. |
| 1. | Most stable dimer (ΔG = −10.4 kcal/mol) |

| | | |
|---|---|---|
| | 5′ GCGAAGCGCGACAIYTCCCA 3′ | (SEQ ID NO:6) |
| | \|\|\|\| | |
| | 3′ ACCCTYIACAGCGCGAAGCG 5′ | (SEQ ID NO:6) |
| 2. | (ΔG = −9.9 kcal/mol) | |
| | 5′ GCGAAGCGCGACAIYTCCCA 3′ | (SEQ ID NO:6) |
| | \| \|\|\|\| \| | |
| | 3′ ACCCTYIACAGCGCGAAGCG 5′ | (SEQ ID NO:6) |
| | No false Priming sites found. | |

Secondary Structure or primer pair

| | |
|---|---|
| 1. | Most stable cross dimer (ΔG = −7.2 kcal/mol) (3′ cross dimer) |

| | |
|---|---|
| 5′ CCGACGACCGACCCCWAYCTGCT 3′ | (SEQ ID NO:5) |
| \| \|\|\| | |
| 3′ ACCCTYIACAGCGCGAAGCG 5′ | (SEQ ID NO:6) |

C. Yersinia group

Sequence Primer Premier alignment
(Sense Primer)

| | | |
|---|---|---|
| | 5′ GTTAGAAATTTTCGAYGTCAG 3′ | (SEQ ID NO:7) |
| | \|\|\|\|\|\|\|\|\|\|\|\|\|\|\| \|\|\|\|\| | |
| Maj. Con. | 3′ (3) CAATCTTTAAAAGCTACAGTC (23) 5′ | (SEQ ID NO:55) |
| Min. Con. | 3′ (3) CRAWCTTKAMAARCTRCAGTC (23) 5′ | (SEQ ID NO:56) |
| Yersinia yukI | 5′ (3) GTTAGAAATTTTCGATGTCAG (23) 3′ | (SEQ ID NO:57) |
| Yersinia ybtI | 5′ (3) GTTAGAAATTTTCGATGTCAG (23) 3′ | (SEQ ID NO:57) |
| E. agglomerans eagI | 5′ (3) GTTAGAAATTTTTGATGTCAG (23) 3′ | (SEQ ID NO:58) |
| E. stewarrtii esaI | 5′ (3) GCTTGAACTGTTTGACGTCAG (23) 3′ | (SEQ ID NO:59) |
| (Antisense Primer) | | |
| | 5′ ATCATACTCATCAAACTCCAT 3′ | (SEQ ID NO:8) |
| | \|\|\|\|\|\|\|\|\|\|\|\|\|\|\|\|\|\|\|\|\| | |
| Maj. Con. | 3′ (124) TAGTATGAGTAGTTTGAGGTA (144) 5′ | (SEQ ID NO:8) |
| Min. Con. | 3′ (124) TAGYWTRAGYAGYYTGAGGTA (144) 5′ | (SEQ ID NO:60) |
| Yersinia yukI | 3′ (124) TAGCATGAGTAGTTTGAGGTA (144) 5′ | (SEQ ID NO:61) |
| Yersinia ybtI | 3′ (124) TAGTATAAGTAGTTTGAGGTA (144) 5′ | (SEQ ID NO:62) |
| E. agglomerans eagI | 3′ (124) TAGTTTGAGCAGCTTGAGGTA (144) 5′ | (SEQ ID NO:63) |
| E. stewartii esaI | 3′ (124) TAGTTTAAGTAGCCTGAGGTA (144) 5′ | (SEQ ID NO:64) |

Properties

| | | | | | | | | |
|---|---|---|---|---|---|---|---|---|
| | | | | | | | | Ta |
| | | | Tm | | ΔG | Activity | | Opt |
| Rating | Seq. No. | Length | (° C.) | GC % | (kcal/mol) | (μg/OD) | Degeneracy | (° C.) |

| | | | | | | | | | |
|---|---|---|---|---|---|---|---|---|---|
| Sense | 84 | 3 | 21 | 0.0 | 35.7 | −35.3 | 31.3 | 2 | — |
| Antisense | 100 | 144 | 21 | 48.3 | 33.3 | −33.8 | 31.3 | 1 | — |
| Product | 10 | — | 142 | 80.3 | 34.5 | — | — | — | 31.2 |

Product Size

| | |
|---|---|
| Yersinia (yukI) | 142 |
| Yersinia (ybtI) | 142 |
| E. agglomerans (eagI) | 142 |
| E. stewartii (esaI) | 142 |

Secondary Structure of Sense Primer

| | |
|---|---|
| | No hairpins found. |
| 1. | Most stable dimer (ΔG = −9.3 kcal/mol) |

| | | |
|---|---|---|
| | 5′ GTTAGAAATTTTCGAYGTCAG 3′ | (SEQ ID NO:7) |
| | \| \|\|\|\|\|\| \| | |
| | 3′ GACTGYAGCTTTTAAAGATTG 5′ | (SEQ ID NO:7) |
| 2. | (ΔG = −9.9 kcal/mol) | |
| | 5′ GTTAGAAATTTTCGAYGTCAG 3′ | (SEQ ID NO:7) |
| | \|\|\|\| | |
| | 3′ GACTGYAGCTTTTAAAGATTG 5′ | (SEQ ID NO:7) |
| 3. | (ΔG = −5.5 kcal/mol) | |
| | 5′ GTTAGAAATTTTCGAYGTCAG 3′ | (SEQ ID NO:7) |
| | \| \|\|\|\| \|\|\|\| \| | |
| | 3′ GACTGYAGCTTTTAAAGATTG 5′ | (SEQ ID NO:7) |
| | No false priming sites found. | |

Secondary Structure of antisense primer

No hairpins found.
No dimers found.
No false priming sites found.

Secondary Structure or primer pair

No cross dimers found.

D. Erwinia group

Sequence Primer Premier alignment
(Sense Primer)

| | | |
|---|---|---|
| | 5′ ATTGAAACGAAATACCCTAMYATG 3′ | (SEQ ID NO:3) |
| | \|\|\|\|\|\|\|\|\|\|\|\|\|\|\|\|\|\|\| \|\|\| | |
| Maj. Con. | 3′ (3) TAACTTTGCTTTATGGGATTGTAC (23) 5′ | (SEQ ID NO:65) |
| Min. Con. | 3′ (3) TAWCTWTRYTTTATGGGATTGTAC (23) 5′ | (SEQ ID NO:66) |
| E. carotovora carI | 5′ (3) ATTGAAACAAAATACCCTAACATG (23) 3′ | (SEQ ID NO:67) |
| E. carotovora expI | 5′ (3) ATTGAAACGAAATATCCTAATATG (23) 3′ | (SEQ ID NO:68) |
| E. chrysanthemi expI | 5′ (3) ATAGAAATGAAATATCCCAACATG (23) 3′ | (SEQ ID NO:69) |
| E. chrysanthemi echI | 5′ (3) ATAGAAATAAAATACCCCAATATG (23) 3′ | (SEQ ID NO:70) |
| Serratia swrI | 5′ (3) ATTGAAACGAAATACCCAAACATG (23) 3′ | (SEQ ID NO:71) |
| Yersinia yenI | 5′ (3) ATTGATATGAAATATCCAACCATG (23) 3′ | (SEQ ED NO:72) |
| (Antisense Primer) | | |
| | 5′ ATTCCCCAGCCAGADCGTT 3′ | (SEQ ID NO:4) |
| | \|\|\|\|\|\|\|\|\|\|\|\|\|\| \|\|\|\| | |
| Maj. Con. | 3′ (458) TAAGGGGTCGGTCTTGCAA (476) 5′ | (SEQ ID NO:73) |
| Min. Con. | 3′ (458) TAHRVGGTYGGHCTHGCAA (476) 5′ | (SEQ ID NO:74) |
| E. carotovora carI | 3′ (455) TAAGGGGTCGGTCTAGCAA (473) 5′ | (SEQ ID NO:75) |
| E. carotovora expI | 3′ (455) TAAAAGGTTGGTCTTGCAA (473) 5′ | (SEQ ID NO:76) |
| E. chrysanthemi expI | 3′ (455) TACGCGGTCGGACTTGCAA (473) 5′ | (SEQ ID NO:77) |
| E. chrysanthemi echI | 3′ (455) TACGCGGTCGGACTTGCAA (473) 5′ | (SEQ ID NO:77) |
| Serratia swrI | 3′ (458) TATACGGTTGGCCTTGCAA (476) 5′ | (SEQ ID NO:78) |
| Yersinia yenI | 3′ (455) TATAAGGTTGGACTCGCAA (473) 5′ | (SEQ ID NO:79) |

Properties

| | | | | | | | | |
|---|---|---|---|---|---|---|---|---|
| | | | | | | | | Ta |
| | | | Tm | | ΔG | Activity | | Opt |
| Rating | Seq. No. | Length | (° C.) | GC % | (kcal/mol) | (μg/OD) | Degeneracy | (° C.) |

| | | | | | | | | | |
|---|---|---|---|---|---|---|---|---|---|
| Sense | 100 | 208 | 24 | 0.0 | 33.3 | −41.5 | 30.1 | 4 | — |
| Antisense | 100 476 | 19 | 0.0 | 54.4 | −39.0 | 32.9 | 3 | — | |
| Product | 90 | — | 269 | 84.0 | 38.7 | — | — | — | 33.8 |

Product Size

| | |
|---|---|
| E. carotovora (carI) | 266 |
| E. carotovora (expI) | 266 |
| E. chrysanthemi (expI) | 266 |
| E. chrysanthemi (echI) | 266 |
| Serratia (swrI) | 269 |
| Yersinia (yenI) | 266 |

Secondary Structure of Sense Primer

No hairpins found.
No dimers found.
No false priming sites found.

Secondary Structure of antisense primer

No hairpins found.
No dimers found.
No false priming sites found.

Secondary Structure or primer pair

| | |
|---|---|
| 1. | Most stable cross dimer (ΔG = −7.9 kcal/mol) (cross dimer) |

| | |
|---|---|
| 5′ ATTGAAACGAAATACCCTAMYATG 3′ | (SEQ ID NO:3) |
| \|\|\|\| \| \| \| | |
| 3′ TTGCDAGACCGACCCCTTA 5′ | (SEQ ID NO:4) |

E. V. harveyi group (primer set 1)

Sequence Primer Premier alignment
(Sense Primer)

| | | |
|---|---|---|
| | 5′ CGGGTTGCGAAAACVATG 3′ | (SEQ ID NO:9) |
| | \|\|\|\|\|\|\|\|\|\|\|\|\|\| \|\|\| | |
| Maj. Con. | 3′ (3) GCCCAACGCTTTTGCTAC (76) 5′ | (SEQ ID NO:80) |
| Min. Con. | 3′ (3) GCVYAMCGNTTTTGBTAC (76) 5′ | (SEQ ID NO:81) |
| E. coli | 5′ (3) CGGGTGGCGAAAACAATG (75) 3′ | (SEQ ID NO:82) |
| Salmonella | 5′ (3) CGGGTTGCAAAAACGATG (53) 3′ | (SEQ ID NO:83) |
| V. Cholera | 5′ (3) CGTGTTGCCAAAACCATG (75) 3′ | (SEQ ID NO:84) |
| V. harveyi | 5′ (3) CGTGTGGCTAAAACGATG (75) 3′ | (SEQ ID NO:85) |
| Haemophilus | 5′ (3) CGCATTGCAAAAACGATG (75) 3′ | (SEQ ID NO:86) |
| (Antisense Primer) | | |
| | 5′ ACAGATGCTCCARIGTATG 3′ | (SEQ ID NO:10) |
| | \|\|\|\|\|\|\|\|\|\|\|\| \|\|\|\|\| | |
| Maj. Con. | 3′ (161) TGTCTACGAGGTCCCATAC (179) 5′ | (SEQ ED NO:87) |
| Min. Con. | 3′ (161) TVTYYACRAGDTYNCATAC (179) 5′ | (SEQ ID NO:88) |
| E. coli | 3′ (160) TGTCCACGAGGTCCCATAC (178) 5′ | (SEQ ED NO:89) |
| Salmonella | 3′ (137) TGTCTACGAGTTCGCATAC (155) 5′ | (SEQ ID NO:90) |
| V. cholera | 3′ (160) TCTCTACGAGATCTCATAC (178) 5′ | (SEQ ID NO:91) |
| V. harveyi | 3′ (159) TGTTTACGAGATTACATAC (177) 5′ | (SEQ ID NO:92) |
| Haemophilus | 3′ (160) TATTTACAAGTTCACATAC (178) 5′ | (SEQ ID NO:93) |

Properties

| | | | | | | | | |
|---|---|---|---|---|---|---|---|---|
| | | | | | | | | Ta |
| | | | Tm | | ΔG | Activity | | Opt |
| Rating | Seq. No. | Length | (° C.) | GC % | (kcal/mol) | (μg/OD) | Degeneracy | (° C.) |

| | | | | | | | | | |
|---|---|---|---|---|---|---|---|---|---|
| Sense | 100 | 59 | 18 | 0.0 | 53.7 | −36.5 | 31.7 | 3 | — |
| Antisense | 100 | 179 | 19 | 0.0 | 44.7 | −32.3 | 31.9 | 8 | — |
| Product | 100 | — | 121 | 83.1 | 43.8 | — | — | — | 33.1 |

Product Size

| | |
|---|---|
| E. coli | 121 |
| Salmonella | 120 |
| V. cholera | 121 |
| V. harveyi | 120 |
| Haemophilus | 121 |

Secondary Structure of Sense Primer

No hairpins found.
No dimers found.
No false priming sites found.

Secondary Structure of antisense primer

No hairpins found.
No dimers found.
No false priming sites found.

Secondary Structure or primer pair

No cross dimers found.

F. V. harveyi group (primer set 2)

Sequence Primer Premier alignment
(Sense Primer)

| | | |
|---|---|---|
| | 5′ CATCTGTTTGCTGGCTTTAT 3′ | (SEQ ID NO:34) |
| | \|\|\|\|\|\|\|\|\|\|\|\|\|\|\|\|\|\|\|\| | |
| Maj. Con. | 3′ (173) GTAGACAAACGACCGAAATA (192) 5′ | (SEQ ID NO:34) |
| Min. Con. | 3′ (173) GTRRABAWRCGHCCDAAATA (192) 5′ | (SEQ ID NO:94) |
| E. coli | 5′ (172) CACCTGTTTGCTGGTTTTAT (191) 3′ | (SEQ ID NO:95) |
| Salmonella | 5′ (149) CATCTGTTTGCTGGCTTTAT (168) 3′ | (SEQ ID NO:96) |
| V. cholera | 5′ (172) CATCTCTACGCGGGCTTTAT (191) 3′ | (SEQ ID NO:97) |
| V. harveyi | 5′ (171) CATTTGTACGCAGGCTTTAT (190) 3′ | (SEQ ID NO:98) |
| Haemophilus | 5′ (172) CATTTATTTGCTGGATTTAT (191) 3′ | (SEQ ID NO:99) |
| (Antisense Primer) | | |
| | 5′ CATCTGGCGYRCCAATCA 3′ | (SEQ ID NO:35) |
| | \|\|\|\|\|\|\|\|\| \|\|\|\|\|\|\| | |
| Maj. Con. | 3′ (273) GTAGACCGCATGGTTAGT (290) 5′ | (SEQ ID NO:100) |
| Min. Con. | 3′ (273) GHMDDCCRCRYGGTTAGT (290) 5′ | (SEQ ID NO:101) |
| E. coli | 3′ (272) GTAGACCGCATGGTTAGT (289) 5′ | (SEQ ID NO:102) |
| Salmonella | 3′ (249) GCAGGCCGCACGGTTAGT (266) 5′ | (SEQ ID NO:103) |
| V. cholera | 3′ (272) GACAGCCGCGTGGTTAGT (289) 5′ | (SEQ ID NO:104) |
| V. harveyi | 3′ (271) GACTTCCGCATGGTTAGT (288) 5′ | (SEQ ID NO:105) |
| Haemophilus | 3′ (272) GTAAACCACACGGTTAGT (289) 5′ | (SEQ ID NO:106) |

Properties

| | | | | | | | | |
|---|---|---|---|---|---|---|---|---|
| | | | | | | | | Ta |
| | | | Tm | | ΔG | Activity | | Opt |
| Rating | Seq. No. | Length | (° C.) | GC % | (kcal/mol) | (μg/OD) | Degeneracy | (° C.) |

| | | | | | | | | | |
|---|---|---|---|---|---|---|---|---|---|
| Sense | 100 | 173 | 20 | 53.2 | 40.0 | −36.6 | 35.1 | 1 | — |
| Antisense | 85 | 290 | 18 | 0.0 | 55.6 | −35.2 | 32.9 | 4 | — |
| Product | 0 | — | — | 84.9 | 48.3 | — | — | — | 34.4 |

Product Size

| | |
|---|---|
| E. coli | 118 |
| Salmonella | 118 |
| V. cholera | 118 |
| V. harveyi | 118 |
| Haemophilus | 118 |

Secondary Structure of Sense Primer

No hairpins found.
No dimers found.
No false priming sites found.

Secondary Structure of antisense primer

| | |
|---|---|
| | No hairpins found. |
| | No dimers found. |
| | Most stable false priming site: |
| | Most stable false priming site: |
| 1. | (ΔG = −9.9 kcal/mol) (3′ false priming site); Product = 0 |

| | |
|---|---|
| 5′ CATCTGGCGYRCCAATCA 3′ | (SEQ ID NO:35) |
| \|\| \|\|\|\| \|\|\|\|\|\| | |
| 3′ (84) GTTTTCCGCTGTGTTAGT (101) 5′ | (SEQ ID NO:107) |

| | |
|---|---|
| 2. | (ΔG = −9.9 kcal/mol) (3′ false priming site); Product = 0 |

| | |
|---|---|
| 5′ CATCTGGCGYRCCAATCA 3′ | (SEQ ID NO:35) |
| \|\| \|\|\|\|\|\| | |
| 3′ (184) ACCGAAATACGCGTTAGT (201) 5′ | (SEQ ID NO:108) |

Secondary structure of primer pair

| | |
|---|---|
| 1. | Most stable cross dimer (ΔG = −9.9 kcal/mol) (3′ cross dimer) |

| | |
|---|---|
| 5′ CATCTGTTTGCTGGCTTTAT 3′ | (SEQ ID NO:34) |
| \|\| \|\|\| | |
| 3′ ACTAACCRYGCGGTCTAC 5′ | (SEQ ID NO:35) |

Preparation of DNA

Bacterial cells used for DNA extraction were grown on agar plates containing media appropriate to each organism. Genomic DNA was extracted and partially purified using commercial kits according to the manufacturer's directions; the primary kits used were DNAZol (Life Technologies, Inc.) and BactoZol (Molecular Research Center, Inc., Cincinnati, Ohio, www.mrcgene.com/). Extraction and purification of DNA from environmental samples were performed using a commercial kit (Ultra Clean Kit, MoBio Corp., Solana Beach, Calif., www.mobio.com/). No further purification of DNA was attempted regardless of source. For the environmental samples and quantitative comparisons among known bacterial samples, DNA concentration was measured by spectroscopy and adjusted to 30 μg ml₋₁ before use.

PCR and Electrophoresis

Target DNA was amplified using a MasterCycler Gradient thermal cycler (Eppendorf, Inc., Westbury, N.Y.) and 20 μL reaction volumes. Reactions consisted of 1 μL of template DNA prepared as described above added to 19 μL of a commercially prepared complete reaction mixture (JumpStart ReadyMix REDTaq DNA Polymerase, Sigma, St. Louis, Mo., catalog number P0982) containing primers at 0.4 μM. Amplification conditions were initial denaturation at 95° C. for 4.5 min followed by 30 cycles of denaturation at 95° C. for 30 sec, annealing at either 50° C. or 60° C. (depending on primers used) for 30 sec, and extension at 72° C. for 30 sec. Final extension was at 72° C. for 4 min.

Products of the PCR were separated and visualized by electrophoresis in gels containing 3% agarose (Amresco, Type I). Gel and running buffer were 0.5×TBE. The entire volume of the reaction was used to load each lane of the gel. A 25-bp DNA step ladder was used for estimating product length (Promega, Madison, Wis., catalog number G4511). Electrophoresis conditions were typically 150 V for between 40 and 120 min depending on the size of the gel. Resulting bands were visualized using ethidium bromide on a transilluminator and then photographed using a handheld camera equipped with polaroid film. Selected bands were recovered from gels (QIAQquick Spin Kit, Qiagen, Valencia, Calif., www.qiagen.com/) and partially sequenced.

Comparative Example

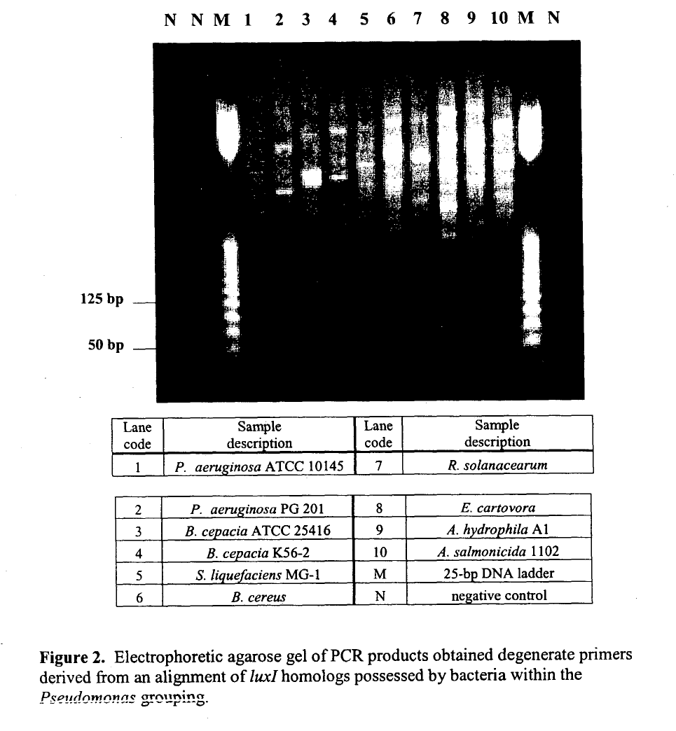

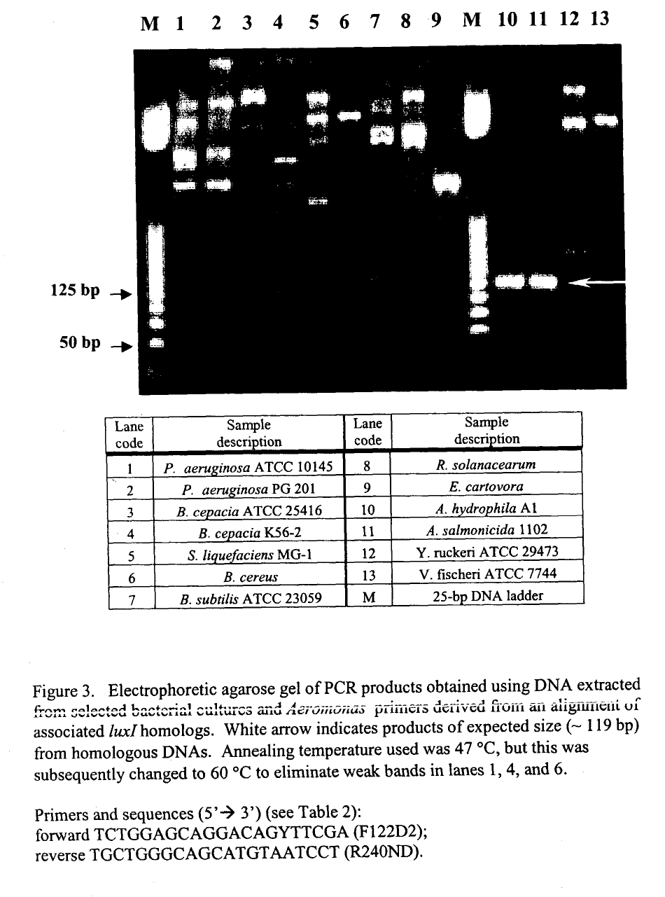

As a comparative example, showing an alternative approach to a PCR-based assay to amplify portions of the luxI gene for detecting quorum sensing potential, a method was performed using primers having degeneracy at various positions throughout the primers (rather than degeneracy solely at the 3′ end). The primers corresponded to sequences for which more than one base was present among the homologs comprising the alignment. The DNA amplified by this approach included an unacceptably high number of non-specific products (FIG. 2).

Results and Discussion

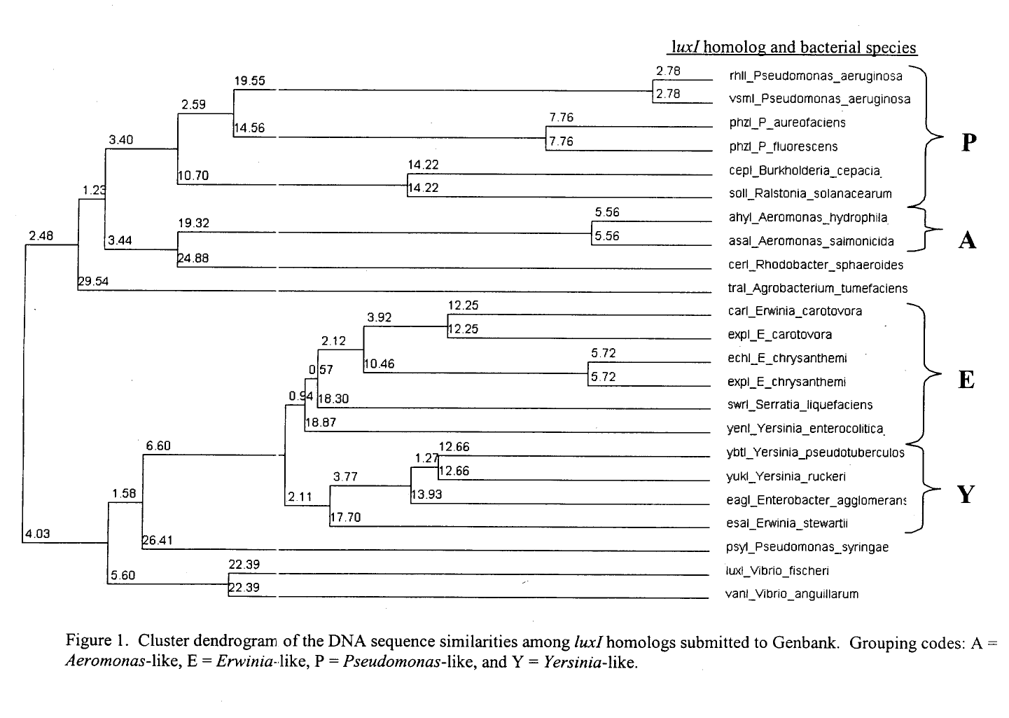

The primers used (Table 2) were designed based on alignments of known full-length sequences of the luxI homologs obtained from Genbank. Alignment of all available sequences of luxI homologs revealed a high degree of diversity among the bacterial taxa and indicated that a minimum of four subgroupings would be necessary to capture the majority of the diversity present and still allow for the designing of effective primers (FIG. 1). Each of the four groups is comprised of bacteria that are taxonomically related: 1) pseudomonads, 2) Aeromonas spp., 3) Erwinia-like species, and 4) Yersinia-like species.

TABLE 2

Sequences and PCR-related information for primers used to detect luxI
homologs associated with selected taxonomic groups
in bacterial communities and pure cultures.

| | | | | |
|---|---|---|---|---|
| | | | | Expected |
| | Primer | | Annealing | nominal product |
| Group | code^{a} | Primer sequence 5′→3′^{b} | Temp. (° C.) | size (bp)^{c} |

| | | | | |
|---|---|---|---|---|
| Aeromonas | F122D2 | TCTGGAGCAGGACAGYTTCGA | 60 | 119 |
| | | (SEQ ID NO:1) | | |
| | R240ND | TGCTGGGCAGCATGTAATCCT | | |
| | | (SEQ ID NO:2) | | |
| Erwinia | F208D4 | ATTGAAACGAAATACCCTAMYATG | 47 | 266 |
| | | (SEQ ID NO:3) | | |
| | R476D3 | ATTCCCCAGCCAGADCGTT | | |
| | | (SEQ ID NO:4) | | |
| Pseudomonas | F295D4 | CCGACGACCGACCCCWAYCTGCT | 60 | 101 |
| | | (SEQ ID NO:5) | | |
| | R398D6^{d} | GCGAAGCGCGACAIYTCCCA | | |
| | | (SEQ ID NO:6) | | |
| Yersinia | F003D2^{e} | GTTAGAAATTTTCGAYGTCAG | 47 | 142 |
| | | (SEQ ID NO:7) | | |
| | R144ND | ATCATACTCATCAAACTCCAT | | |
| | | (SEQ ID NO:8) | | |

^{a}F and D indicate forward and reverse primers, respectively. The three-digit number that follows indicates the location of the 5′ end of the primer within the aligned sequences. ND indicates the primer is non-degenerate. The numeral following the letter D in the remaining codes gives the degree of degeneracy present in the primer, as provided by Primer Premier software.
^{b}Degenerate base codes: Y = C + T, I = deoxyinosine, D = A + T + G, M = A + C, W = A + T.
^{c}Actual size of product may vary ˜± 3 bp
^{d}Vial from Life Technologies is incorrectly labeled R398D8
^{e}Vial from Life Technologies is incorrectly labeled F03D2

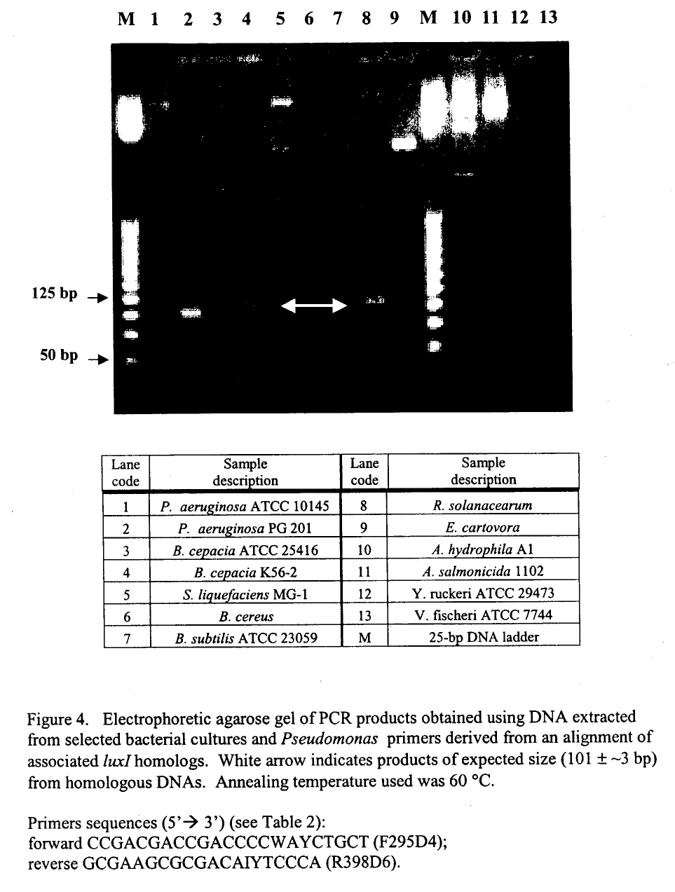

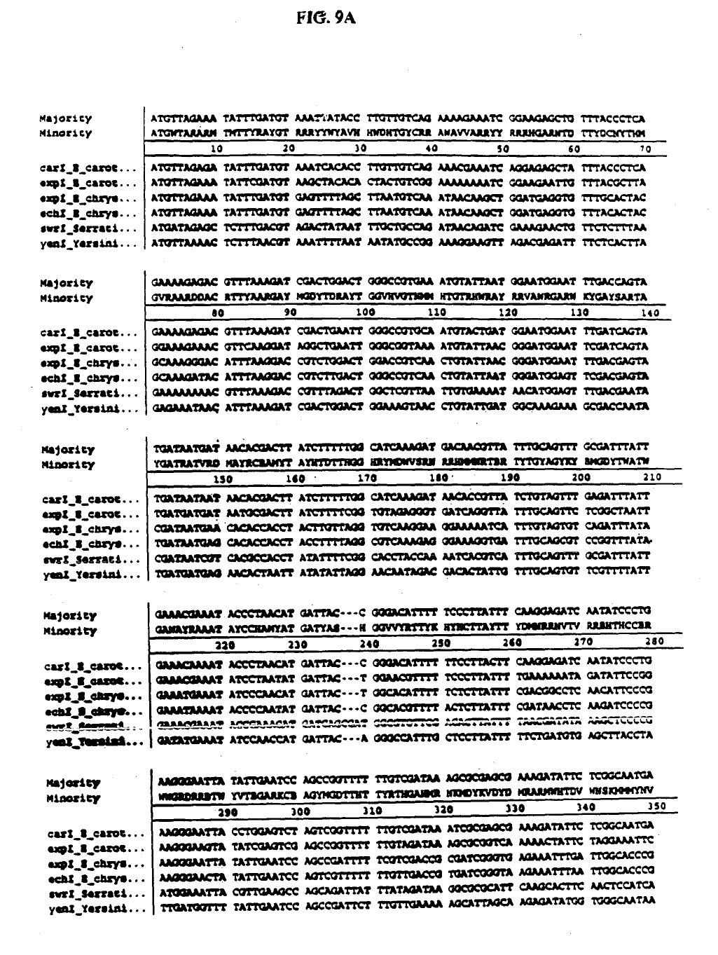

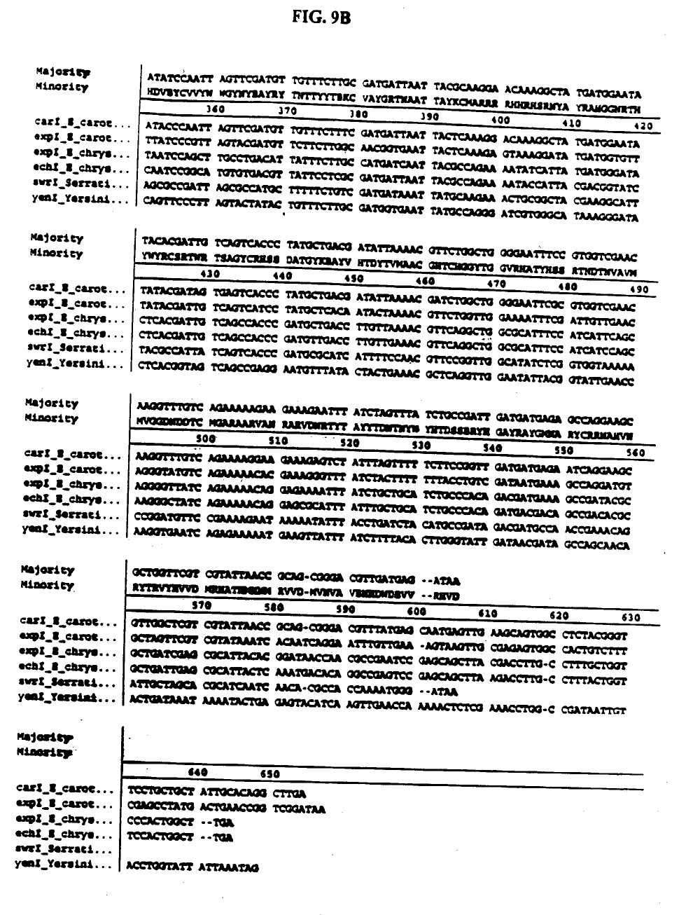

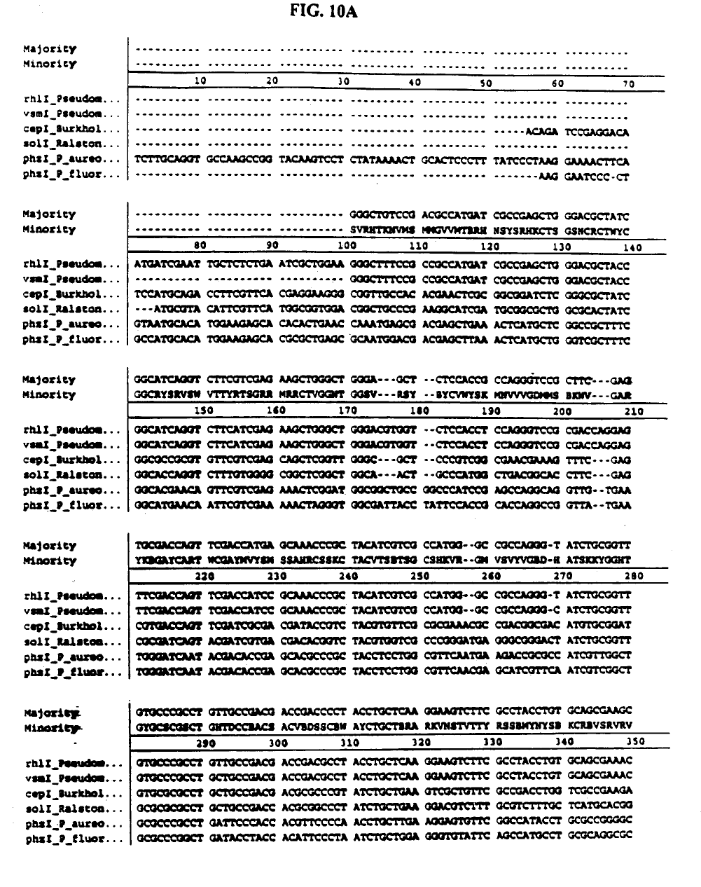

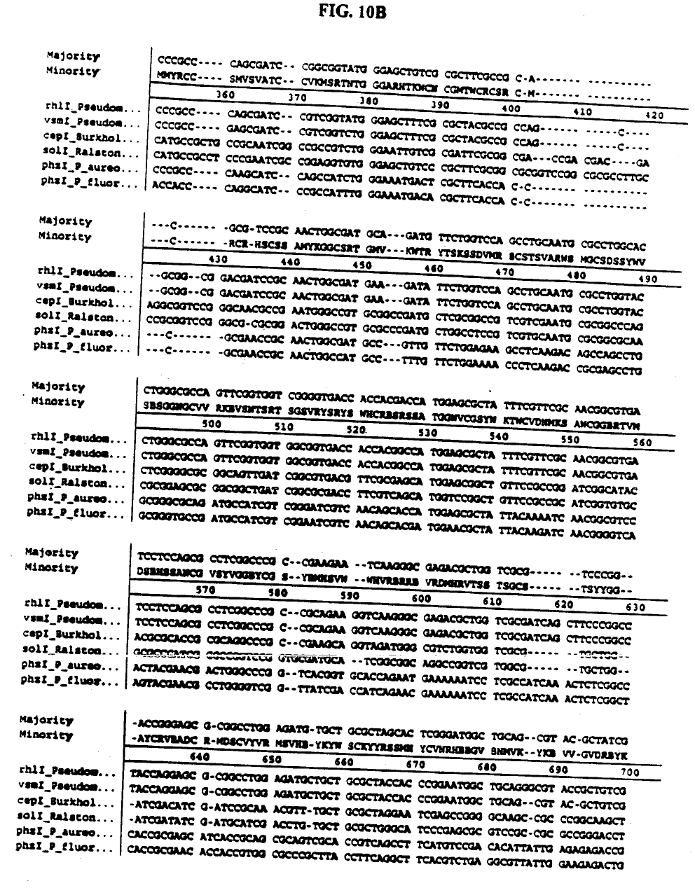

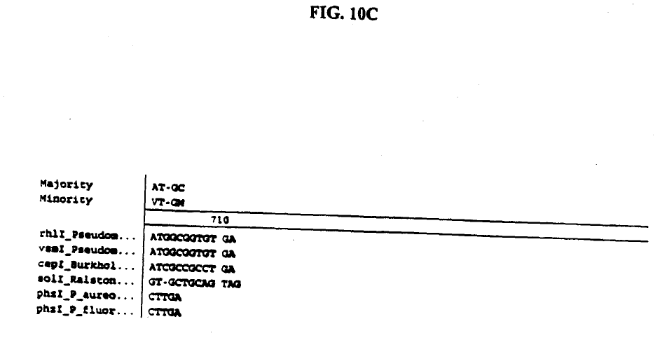

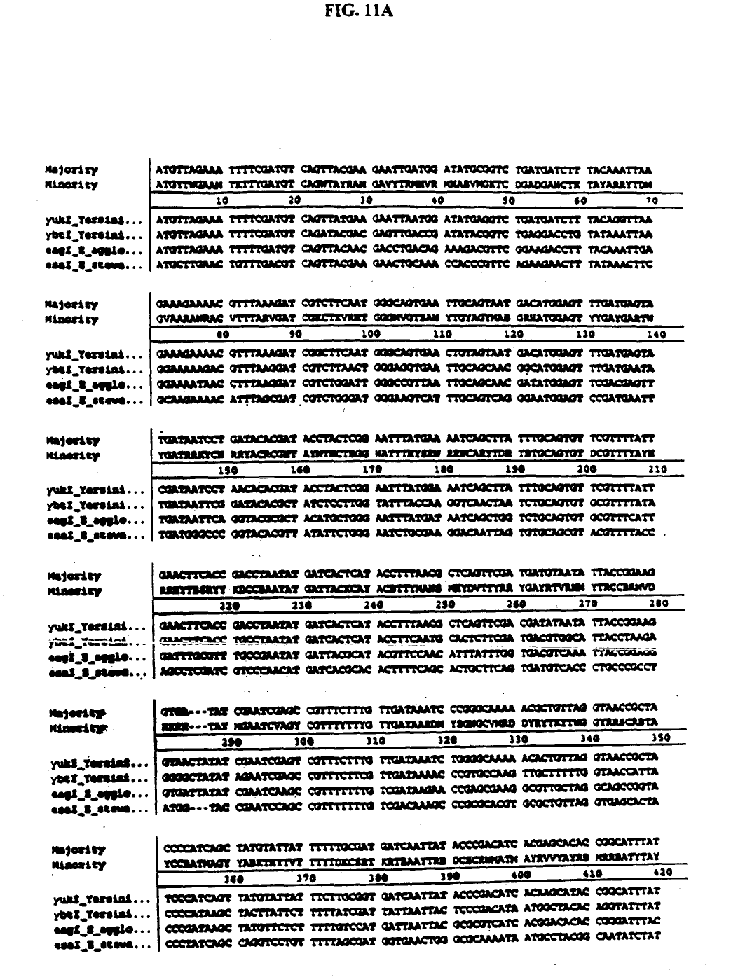

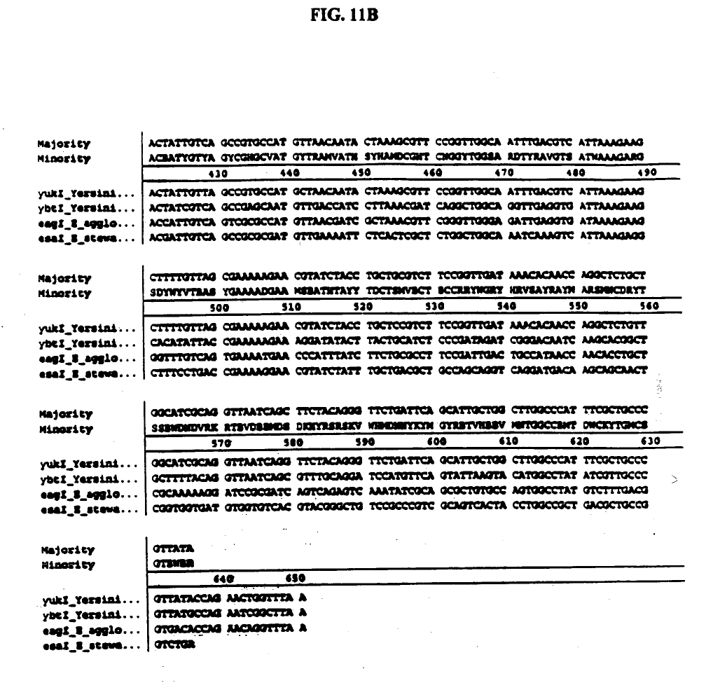

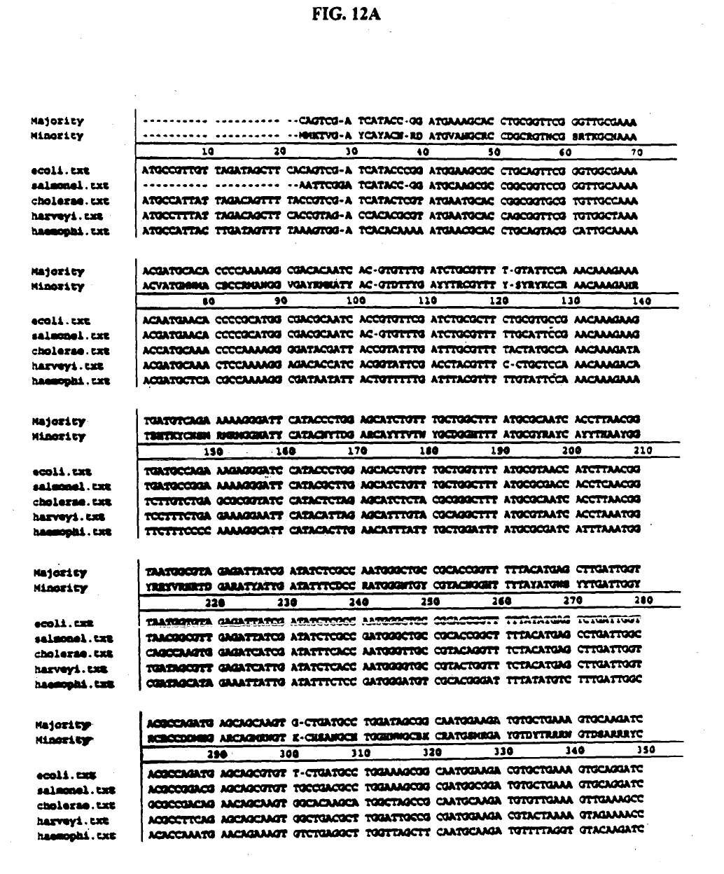

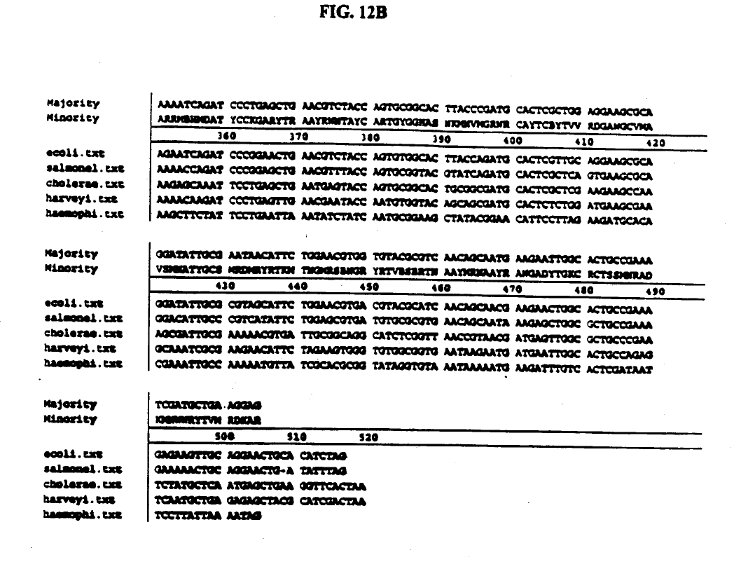

Given this diversity of sequences among the various homologs, early efforts to develop primers focused on the pseudomonad cluster (group P in FIG. 1) as a model for approaching the remaining three groups. Various strategies were employed in designing primers for amplifying the luxI homologs prior to arriving at the sequences eventually adopted (Table 2). Most notably, one early attempt used primers that were degenerate various positions throughout the primer sequences for which more than one base was present among the homologs comprising the alignment (Comparative Example). The DNA amplified by this approach included an unacceptably high number of non-specific products (FIG. 2). The strategy eventually adopted was to identify locations within the alignment for which at minimum the first three bases at the 3′ end of the primer were conserved across all homologs and for which degeneracy of the rest of the primer sequence was low, especially towards the 3′ end of the primer. Additionally, the adopted primers were degenerate only for those base positions at which the alignment was degenerate at positions within the nucleobase positions 4-8 from the 3′ end of the primer; the remainder of the primer sequence was non-degenerate and based on the majority consensus of the alignment. Using this approach, four sets of primers were developed (Table 2 and FIGS. 8-11). These primers successfully amplified DNAs of the proper size for the Pseudomonas and Aeromonas groupings (FIGS. 3-4). The corresponding amplifications for the Yersinia and Erwinia groupings also produced expected products (data not shown). Subsequent work focused on the first two groupings. It is interesting to note the presence of weak bands of proper size for the two Bacillus species when using the Aeromonas primers. Although these were eliminated when the annealing temperature was increased from 47° C. to 60° C., the bands may indicate the presence of a DNA sequence weakly homologous to Aeromonas luxI homologs in these Gram positive bacteria (which are not thought to possess AHL-based quorum sensing).

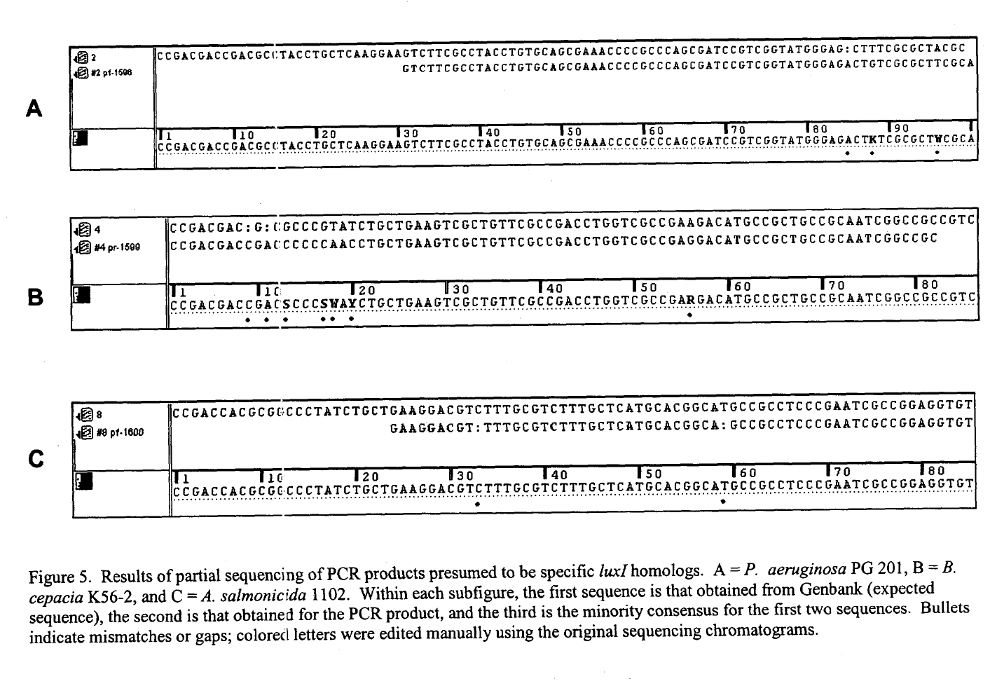

Verification of the PCR products as being the intended luxI homologs was assessed by partial sequencing of DNA amplified from Pseudomonas aeruginosa PG201, Burkholdaria cepacia K56-2, Ralstonia solanacearum (strain identification uncertain, but the culture was obtained from M. Schell who submitted the Genbank sequence for the solI gene included in alignments (Table 1)), and Aeromonas salmonicida 1102. The analyses indicated strongly that the intended sequences were amplified for the first three bacteria listed above (FIG. 5); The high degree of homology between the two sequences used in the alignment for A. salmonicida and the robust production of a fragment of expected size (Table 2, FIG. 3) support the position that the correct sequence was amplified by the PCR. Inconsistencies between the Genbank sequences and the sequencing results should be viewed from the recognition that the coverage reported was only at a 1× level, whereas it is normally recommended that a minimum of 6× coverage be used to help resolve such inconsistencies.

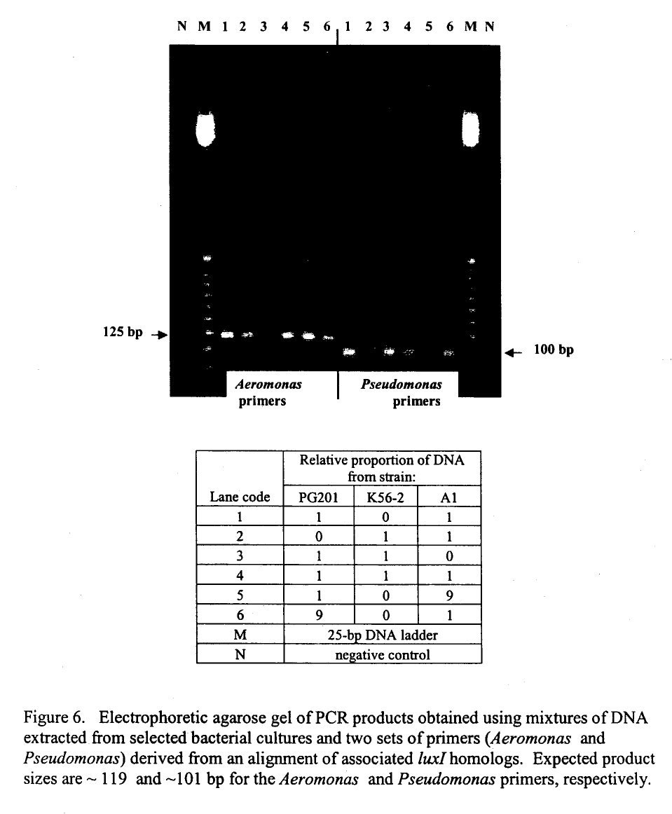

An evaluation of the utility of the PCR protocols to amplify intended targets in the presence of known background DNA was performed by mixing together, in various proportions, genomic DNAs of Pseudomonas aeruginosa PG201, Burkholdaria cepacia K56-2, and Aeromonas hydrophila A1 (FIG. 6). The Aeromonas and Pseudomonas primers both worked well when the homologous and non-homologous DNA were present in a 1:1 ratio. However, only the former performed adequately when the target DNA constituted 10% of the total DNA present in the reaction. This probably reflects in part the relatively high degree of homology between the Aeromonas target DNA and the corresponding primers when compared to those for the Pseudomonas grouping. It should be noted that the weak band present for Aeromonas lane 3 probably resulted from contamination, as an apparently identical band was observed in the corresponding negative control lane.

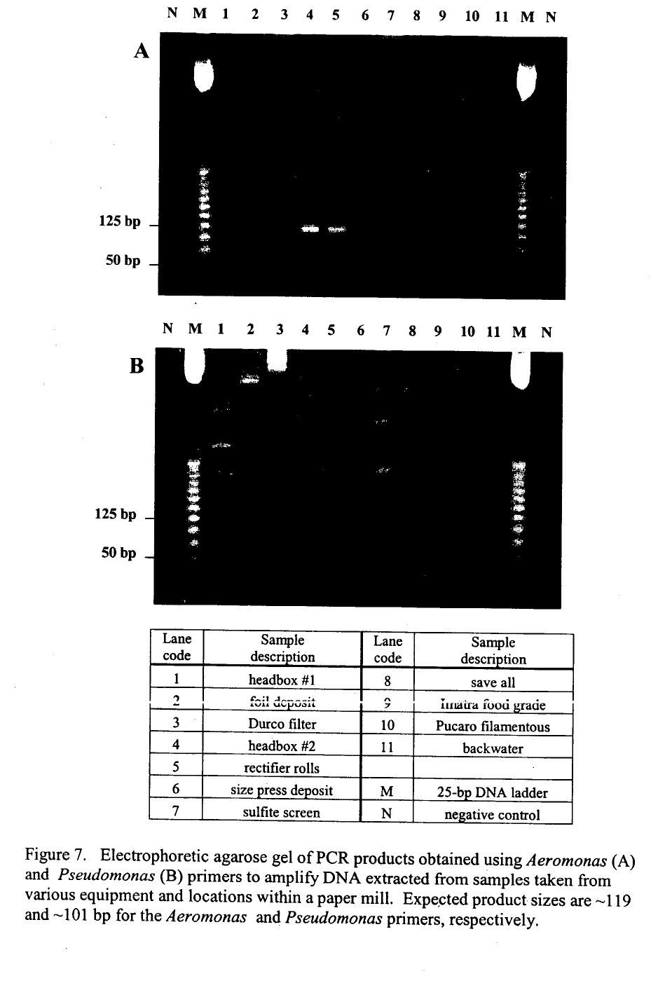

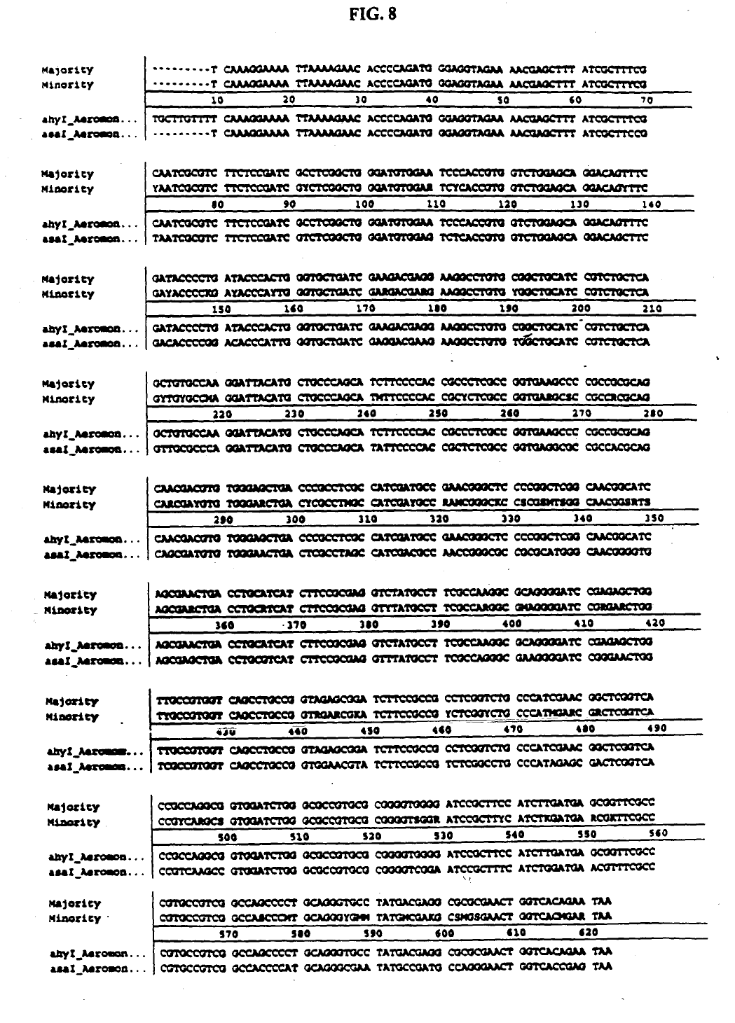

An initial assessment was made of the ability of the PCR to amplify any intended targets present in environmental DNA samples taken from various locations and equipment in a paper mill (FIG. 7). The Aeromonas primers detected the presence of homologous DNA in samples from the rectifier rolls, sulfite screen and both headboxes. The Pseudomonas primers gave positive results for the same four samples and also for the Durco filter (weak band), save all, and Pucaro filamentous samples. The foil and size press deposits and the Imatra food grade and back water samples gave negative results for both sets of primers.

The results described here are of great significance, as they indicate that by using primers selective for particular genetic groupings, an assessment of the taxonomic composition of the luxI homolog-bearing members within a bacterial community may be made. In turn, this information could be of value in developing strategies to selectively manipulate those bacterial taxa present in a community that possess luxI homologs in their genomes. It should also be noted that this general approach to assessing quorum sensing potentials has potential application to other quorum sensing systems such as those involving homologs of the luxS gene of Vibrio harveyi.

The PCR-based method of the invention has been developed to assess the quorum sensing potentials of bacterial communities in environmental samples. The fundamental concept is to use PCR primers to amplify sequences of certain lengths from luxI gene homologs present among members of a microbial community. As noted above, the luxI homologs produce the autoinducing AHSLs that trigger quorum sensing events in many Gram-negative bacteria. As such, the method described is gives an indication of the potential for quorum sensing to be active within a community.

What is claimed is:

1. A method of detecting quorum sensing potential of a microorganism in a sample comprising:
(a) extracting nucleic acid from a sample comprising at least one type of microorganism;
(b) performing a polymerase chain reaction using said nucleic acid wherein said polymerase chain reaction comprises a first oligonucleotide primer and a second oligonucleotide primer, wherein each of said primers comprises at least 15 nucleobases, wherein said primers anneal to a consensus sequence of a lux gene and homologs thereof, wherein the first eight nucleobases at the 3′ end of said primers comprise a match to said consensus sequence with optional degeneracy at positions 4-8 from the 3′ end of said primers; wherein said polymerase chain reaction results in amplified DNA fragments;
(c) separating said amplified DNA fragments; and
(d) determining whether any amplified DNA fragment corresponds to the size of a predicted product related to quorum sensing potential, thereby detecting quorum sensing potential of said microorganism in said sample.

2. The method of claim 1 wherein said lux gene is selected from the group consisting of luxI, luxR, and luxS.

3. The method of claim 1 wherein said microorganism is a bacterium.

4. The method of claim 3 wherein said bacterium is a Gram-negative bacterium.

5. The method of claim 1 wherein said sample is taken from the environment.

6. The method of claim 1 wherein said separating is performed on a gel.

7. The method of claim 1 wherein said separating is performed by liquid chromatography.

8. The method of claim 2 wherein said lux gene is the luxI gene.

9. The method of claim 8 wherein said first oligonucleotide primer comprises SEQ ID NO: 1 and said second oligonucleotide primer comprises SEQ ID NO: 2.

10. The method of claim 8 wherein said first oligonucleotide primer comprises SEQ ID NO: 3 and said second oligonucleotide primer comprises SEQ ID NO: 4.

11. The method of claim 8 wherein said first oligonucleotide primer comprises SEQ ID NO: 5 and said second oligonucleotide primer comprises SEQ ID NO: 6.

12. The method of claim 8 wherein said first oligonucleotide primer comprises SEQ ID NO: 7 and said second oligonucleotide primer comprises SEQ ID NO: 8.

13. The method of claim 1 wherein said lux gene is the luxS gene.

14. The method of claim 13 wherein said first oligonucleotide primer comprises SEQ ID NO: 9 and said second oligonucleotide primer comprises SEQ ID NO: 10.

15. A composition comprising
(a) a nucleic acid sequence comprising a lux gene or homolog thereof;
(b) at least two oligonucleotide primers, wherein each of said primers comprises at least 15 nucleobases, wherein said primers anneal to a consensus sequence of a lux gene and homologs thereof; wherein the first eight nucleobases at the 3′ end of said primers comprise a match to said consensus sequence with optional degeneracy at positions 4-8 from the 3′ end of said primers;
(c) at least one enzyme having DNA polymerase activity.

16. The composition of claim 15 further comprising a mixture of deoxynucleotide triphosphates.

17. The composition of claim 15 wherein said primers comprise the sequences of SEQ ID NO: 1 and SEQ ID NO: 2.

18. The composition of claim 15 wherein said primers comprise the sequences of SEQ ID NO: 3 and SEQ ID NO: 4.

19. The composition of claim 15 wherein said primers comprise the sequences of SEQ ID NO: 5 and SEQ ID NO: 6.

20. The composition of claim 15 wherein said primers comprise the sequences of SEQ ID NO: 7 and SEQ ID NO: 8.

21. The composition of claim 15 wherein said primers comprise at least one pair of primers selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; and SEQ ID NO: 9 and SEQ ID NO: 10.

22. A method of amplifying a fragment of a lux gene or homolog thereof comprising:
(a) extracting nucleic acid from a sample comprising at least one type of microorganism, said nucleic acid comprising a lux gene or homolog thereof;
(b) performing a polymerase chain reaction using said nucleic acid wherein said polymerase chain reaction comprises a first oligonucleotide primer and a second oligonucleotide primer, wherein each of said primers comprises at least 15 nucleobases, wherein said primers anneal to a consensus sequence of a lux gene and homologs thereof; wherein the first eight nucleobases at the 3′ end of said primers comprise a match to said consensus sequence with optional degeneracy at positions 4-8 from the 3′ end of said primers; wherein said polymerase chain reaction results in amplified DNA fragments.

23. The method of claim 22 further comprising isolating an amplified fragment of said lux gene or homolog thereof.

24. The method of claim 22 wherein said primers are selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; and SEQ ID NO: 9 and SEQ ID NO: 10.

25. The isolated amplified fragment of claim 23.

26. The method of claim 24 further comprising isolating an amplified fragment of said lux gene or homolog thereof.

27. The isolated amplified fragment of claim 26.

28. A kit for the amplification of a portion of a lux gene, or homolog thereof comprising, in separate containers:
(a) a polymerase,
(b) a plurality of deoxynucleotide triphosphates;
(c) a first oligonucleotide primer; and
(d) a second oligonucleotide primer;
wherein each of said primers comprises at least 15 nucleobases, wherein said primers anneal to a consensus sequence of a lux gene and homologs thereof; and wherein the first eight nucleobases at the 3′ end of said primers comprise a match to said consensus sequence with optional degeneracy at positions 4-8 from the 3′ end of said primers.

29. The kit of claim 28 wherein said kit first oligonucleotide primer comprises SEQ ID NO: 1 and said second oligonucleotide primer comprises SEQ ID NO: 2.

30. The kit of claim 28 wherein said kit first oligonucleotide primer comprises SEQ ID NO: 3 and said second oligonucleotide primer comprises SEQ ID NO: 4.

31. The kit of claim 28 wherein said kit first oligonucleotide primer comprises SEQ ID NO: 5 and said second oligonucleotide primer comprises SEQ ID NO: 6.

32. The kit of claim 28 wherein said kit first oligonucleotide primer comprises SEQ ID NO: 7 and said second oligonucleotide primer comprises SEQ ID NO: 8.

33. The kit of claim 28 wherein said kit first oligonucleotide primer comprises SEQ ID NO: 9 and said second oligonucleotide primer comprises SEQ ID NO: 10.
